(19) United States
(12) Patent Application Publication
(10) Pub. No.: US 2020/0199104 A1
(43) Pub. Date: Jun. 25, 2020

(54) Novel Polymorphic Forms of a TGFBeta Inhibitor

(71) Applicant: PFIZER INC., New York, NY (US)

(72) Inventors: Anand Venkataramana Sistla, Carlsbad, CA (US); Iain David Roy, Rosyth (GB); Andrew Robbins, Middlewich (GB)

(73) Assignee: PFIZER INC., New York, NY (US)

(21) Appl. No.: 16/716,696

(22) Filed: Dec. 17, 2019

Related U.S. Application Data
(60) Provisional application No. 62/782,411, filed on Dec. 20, 2018.
(60) Provisional application No. 62/930,170, filed on Nov. 4, 2019.

(51) Int. Cl. C07D 401/12

(52) U.S. Cl. CPC C07D 401/12; C07B 2200/13

(57) ABSTRACT

The present invention relates to novel crystalline polymorphic and amorphous form of 4-(2-(5-chloro-2-fluorophenyl) -5-isopropylpyridin-4-ylamino)-N-(1,3-dihydroxypropan-2-yl) nicotinamide and to methods for their preparation; and the invention is also directed to pharmaceutical compositions containing at least one polymorphic form and to the therapeutic or prophylactic use of such polymorphic forms and compositions.

### FIELD OF THE INVENTION

The present invention relates to novel polymorphic forms of 4-(2-(5-chloro-2-fluorophenyl) -5-isopropylpyridin-4-ylamino)-N-(1,3-dihydroxypropan-2-yl)nicotinamide, or salts thereof, or hydrates or solvates of any thereof, and to methods for their preparation. The invention is also directed to pharmaceutical compositions containing at least one polymorphic form of 4-(2-(5-chloro-2-fluorophenyl)-5-isopropylpyridin-4-ylamino)-N-(1,3-dihydroxypropan-2-yl) nicotinamide, to the therapeutic or prophylactic use of such pharmaceutical compositions, to such pharmaceutical compositions useful as medicaments, and to such pharmaceutical compositions used in the treatment of abnormal cell growth such as cancers in mammals, especially humans.

### BACKGROUND OF THE INVENTION

The compound 4-(2-(5-chloro-2-fluorophenyl)-5-isopropylpyridin-4-ylamino)-N-(1,3-dihydroxypropan -2-yl)nicotinamide (also referred to as “Compound 1”):

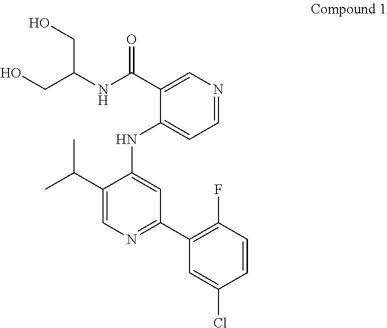 [SMILES: CC(C)c1cnc(-c2cc(Cl)ccc2F)cc1Nc1ccncc1C(=O)NC(CO)CO]

is known to be useful in the treatment of abnormal cell growth, such as cancer, in mammals.

Compound 1, pharmaceutically acceptable salts of Compound 1, and methods of making Compound 1, are described in International Patent application WO2015/103355, and counterpart patent applications such as U.S. patent application Ser. No. 15/109,013, issued as U.S. Pat. No. 10,030,004. A method of making Compound 1 is provided at Example 22 of the '004 patent).

Compound 1 is a potent and selective inhibitor of transforming growth factor-beta (TGFβ). TGFβ belongs to a superfamily of multifunctional proteins that includes, for example, TGFβ1, TGFβ2, and TGFβ3, which are pleiotropic modulators of cell growth and differentiation, embryonic and bone development, extracellular matrix formation, hematopoiesis, and immune and inflammatory responses (Roberts and Sporn Handbook of Experimental Pharmacology (1990) 95:419-58; Massague, et al., Ann. Rev. Cell. Biol. (1990) 6:597-646). For example, TGFβ1 inhibits the growth of many cell types, including epithelial cells, but stimulates the proliferation of various types of mesenchymal cells. Other members of this superfamily include activin, inhibin, bone morphogenic protein, and Mullerian inhibiting substance. The members of the TGFβ family initiate intracellular signaling pathways leading ultimately to the expression of genes that regulate the cell cycle, control proliferative responses, or relate to extracellular matrix proteins that mediate outside-in cell signaling, cell adhesion, migration and intercellular communication. Therefore, inhibitors of the TGFβ intracellular signaling pathway are recognized as being useful primarily for the treatment of fibroproliferative diseases. Fibroproliferative diseases include kidney disorders associated with unregulated TGFβ activity and excessive fibrosis including glomerulonephritis (GN), such as mesangial proliferative GN, immune GN, and crescentic GN. Other renal conditions include diabetic nephropathy, renal interstitial fibrosis, renal fibrosis in transplant patients receiving cyclosporin, and HIV-associated nephropathy. Collagen vascular disorders include progressive systemic sclerosis, polymyositis, scleroderma, dermatomyositis, eosinophilic fasciitis, morphea, or those associated with the occurrence of Raynaud's syndrome. Lung fibroses resulting from excessive TGFβ activity include adult respiratory distress syndrome, chronic obstructive pulmonary disease (COPD), idiopathic pulmonary fibrosis, and interstitial pulmonary fibrosis often associated with autoimmune disorders, such as systemic lupus erythematosus and scleroderma, chemical contact, or allergies. Another autoimmune disorder associated with fibroproliferative characteristics is rheumatoid arthritis. Fibroproliferative conditions can be associated with surgical eye procedures. Such procedures include retinal reattachment surgery accompanying proliferative vitreoretinopathy, cataract extraction with intraocular lens implantation, and post glaucoma drainage surgery. In addition, members of the TGFβ family are associated with the progression of various cancers, M. P. de Caestecker, E. Piek, and A. B. Roberts, J. National Cancer Inst., 92(17), 1388-1402 (2000) and members of the TGFβ family are expressed in large amounts in many tumors. Derynck, Trends Biochem. Sci., 1994, 19, 548-553. For example, it has been found that TGFβ1 inhibits the formation of tumors, probably by inhibition of the proliferation of non-transformed cells. However, once a tumor forms, TGFβ1 promotes the growth of the tumor. N. Dumont and C. L. Arteaga, Breast Cancer Res., Vol. 2, 125-132 (2000). Thus, inhibitors of the TGFβ pathway are also recognized as being useful for the treatment of many forms of cancer, such as lung cancer, skin cancer, and colorectal cancer. In particular, they are considered to be useful for the treatment of cancers of the breast, pancreas, and brain, including glioma.

As understood by those skilled in the art, it is desirable to have crystalline or amorphous forms that possess physical properties amenable to reliable formulation and manufacture. Such properties include filterability, hygroscopicity, and flow, as well as stability to heat, moisture, and light.

Polymorphs are different crystalline forms of the same compound. The term polymorph may or may not include other solid state molecular crystalline forms including hydrates (e.g., bound water present in the crystalline structure) and solvates (e.g., bound solvents other than water) of the same compound. Different crystalline polymorphs typically have different crystal structures due to a different packing of the molecules in the lattice. This results in a different crystal symmetry and/or unit cell parameters which directly influence its physical properties such as the X-ray diffraction characteristics of crystals or powders.

Polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since impurities present may produce undesired toxicological effects. Certain polymorphic forms may also exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

The discussion of the background to the invention herein is included to explain the context of the present invention. This is not to be taken as an admission that any of the material referred to was published, known, or part of the common general knowledge in any country as of the priority date of any of the claims.

### SUMMARY OF THE INVENTION

Two polymorphs of Compound 1 have been identified. Each polymorphic form can be uniquely identified by several different analytical parameters, alone or in combination, such as, but not limited to, powder X-ray diffraction pattern peaks or combinations of two or more peaks; solid state NMR ¹³C and/or ¹⁹F chemical shifts or combinations of two or more chemical shifts; Raman shift peaks or combinations of two or more Raman shift peaks; single crystal unit cell dimensions; or combinations thereof.

One aspect of the present invention provides a crystalline form of 4-(2-(5-chloro-2-fluorophenyl) -5-isopropylpyridin-4-ylamino)-N-(1,3-dihydroxypropan-2-yl)nicotinamide represented as Compound 1:

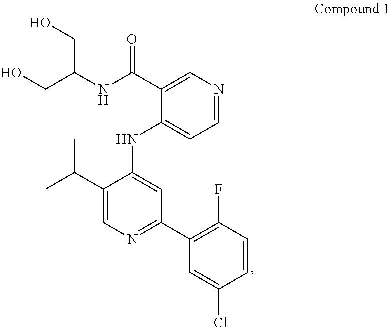 [SMILES: CC(C)c1cnc(-c2cc(Cl)ccc2F)cc1Nc1ccncc1C(=O)NC(CO)CO]

wherein said crystalline form is an anhydrous mono hydrochloride and is the polymorph Form 1. Embodiments of the invention where Compound 1 is Form 1 include those discussed here.

For example, in one embodiment, the present invention provides a crystalline form of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2.

In another embodiment, the present invention provides a crystalline Form 1 of Compound 1 anhydrous mono hydrochloride, wherein said crystalline Form 1 has a Raman spectrum comprising Raman shift peaks (cm−1) at 1594±2 cm⁻¹, 1606±2 cm⁻and 1637±2 cm⁻¹.

In another embodiment, the present invention provides a crystalline Form 1 of Compound 1 anhydrous mono hydrochloride, wherein said crystalline Form 1 has a Raman spectrum comprising Raman shift peaks (cm−1) at 876±2 cm⁻¹, 1519±2 cm⁻¹, 1594±2 cm⁻¹, 1606±2 cm^{-l}and 1637±2 cm⁻¹ .

In another embodiment, the present invention provides a crystalline Form 1 of Compound 1 anhydrous mono hydrochloride, wherein said crystalline Form 1 has a solid state NMR spectrum comprising ¹³C chemical shifts at 136.9±0.2, 26.1±0.2 and 147.7±0.2 ppm.

In another embodiment, the present invention provides a crystalline Form 1 of Compound 1 anhydrous mono hydrochloride, wherein said crystalline Form 1 has a solid state NMR spectrum comprising ¹³C chemical shifts at 136.9±0.2, 26.1±0.2, 147.7±0.2, 125.5±0.2 and 55.4±0.2 ppm.

In another embodiment, the present invention provides a crystalline Form 1 of Compound 1 anhydrous mono hydrochloride, wherein said crystalline Form 1 has a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a powder X-ray diffraction pattern comprising peaks at positions essentially the same as shown in FIG. 1.

In another embodiment, the present invention provides a crystalline Form 1 of Compound 1 anhydrous mono hydrochloride, wherein said crystalline Form 1 has a Raman spectrum comprising Raman shift peaks (cm−1) at positions essentially the same as shown in FIG. 4.

In another embodiment, the present invention provides a crystalline Form 1 of Compound 1 anhydrous mono hydrochloride, wherein said crystalline Form 1 has a solid state NMR spectrum comprising ¹³C chemical shifts at positions essentially the same as shown in FIG. 2.

In another embodiment, the present invention provides a crystalline Form 1 of Compound 1 anhydrous mono hydrochloride, wherein said crystalline Form 1 has a solid state NMR spectrum comprising an ¹⁹F chemical shift at position(s) essentially the same as shown in FIG. 3.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2, and a Raman spectrum comprising Raman shift peaks (cm−1) at at least one of 1594±2 cm⁻¹, 1606±2 cm⁻¹ and 1637±2 cm⁻¹.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2, and a Raman spectrum comprising Raman shift peaks (cm−1) at positions essentially the same as shown in FIG. 4.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2, and a solid state NMR spectrum comprising ¹³C chemical shifts at at least one of 136.9±0.2, 26.1±0.2 and 147.7±0.2 ppm.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2, and a solid state NMR spectrum comprising ¹³C chemical shifts at positions essentially the same as shown in FIG. 2.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2, and a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2, and a solid state NMR spectrum comprising ¹⁹F chemical shifts at position(s) essentially the same as shown in FIG. 3.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of at least one of 13.7±0.2 and 24.4±0.2, and a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a Raman spectrum comprising Raman shift peaks (cm−1) at at least one of 1594±2 cm^{31 1}, 1606±2 cm⁻¹ and 1637±2 cm⁻¹, and a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a solid state NMR spectrum comprising ¹³C chemical shifts at at least one of 136.9±0.2, 26.1±0.2 and 147.7±0.2 ppm, and a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2, a Raman spectrum comprising Raman shift peaks (cm−1) at at least one of 1594±2 cm⁻¹, 1606±2 cm⁻¹ and 1637±2 cm⁻¹, and a solid state NMR spectrum comprising ¹³C chemical shifts at at least one of 136.9±0.2, 26.1±0.2 and 147.7±0.2 ppm.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2, a Raman spectrum comprising Raman shift peaks (cm−1) at at least one of 1594±2 cm⁻¹, 1606±2 cm⁻¹ and 1637±2 cm⁻¹, and a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2, a solid state NMR spectrum comprising ¹³C chemical shifts at at least one of 136.9±0.2, 26.1±0.2 and 147.7±0.2 ppm, and a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of at least one of 13.7±0.2 and 24.4±0.2, a Raman spectrum comprising Raman shift peaks (cm−1) at at least one of 1594±2 cm⁻¹, 1606±2 cm⁻¹ and 1637±2 cm⁻¹, and a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of at least one of 13.7±0.2 and 24.4±0.2, a solid state NMR spectrum comprising ¹³C chemical shifts at at least one of 136.9±0.2, 26.1±0.2 and 147.7±0.2 ppm, and a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a Raman spectrum comprising Raman shift peaks (cm−1) at at least one of 1594±2 cm⁻¹, 1606±2 cm⁻¹ and 1637±2 cm⁻¹, a solid state NMR spectrum comprising ¹³C chemical shifts at at least one of 136.9±0.2, 26.1±0.2 and 147.7±0.2 ppm, and a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of 13.7±0.2 and 24.4±0.2, a Raman spectrum comprising Raman shift peaks (cm−1) at at least one of 1594±2 cm⁻¹, 1606±2 cm⁻¹ and 1637±2 cm⁻¹, a solid state NMR spectrum comprising ¹³C chemical shifts at at least one of 136.9±0.2, 26.1±0.2 and 147.7±0.2 ppm, and a solid state NMR spectrum comprising an ¹⁹F chemical shift at −115.6±0.2 ppm.

In another embodiment, the present invention provides a crystalline Form 1 of Compound 1 anhydrous mono hydrochloride, wherein said crystalline Form 1 has a powder X-ray diffraction pattern comprising peaks at diffraction angle (2 degrees θ) of at least one of 13.7±0.2 and 24.4±0.2, a Raman spectrum comprising Raman shift peaks (cm−1) at at least one of 1594±2 cm⁻¹, 1606±2 cm⁻¹ and 1637±2 cm⁻¹, a solid state NMR spectrum comprising ¹³C chemical shifts at at least one of 136.9±0.2, 26.1±0.2 and 147.7±0.2 ppm, and a solid state NMR spectrum comprising an ¹⁹F chemical shift at −115.6±0.2 ppm.

Another aspect of the present invention provides a crystalline form of 4-(2-(5-chloro-2-fluorophenyl) -5-isopropylpyridin-4-ylamino)- N-(1, 3-dihydroxypropan-2-yl) nicotinamide, represented as Compound 1:

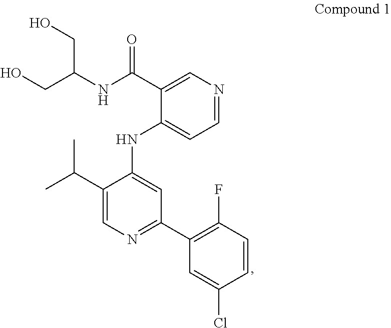 [SMILES: CC(C)c1cnc(-c2cc(Cl)ccc2F)cc1Nc1ccncc1C(=O)NC(CO)CO]

wherein said crystalline form is a channel hydrate mono hydrochloride and is the polymorph Form 2. Embodiments of the invention where Compound 1 is Form 2 include those listed here:

For example, in one embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2, 17.4±0.2, 18.9±0.2 and 28.4±0.2.

In another embodiment, the present invention provides a crystalline Form 2 of Compound 1 channel hydrate mono hydrochloride, wherein said crystalline Form 2 has a Raman spectrum comprising Raman shift peaks (cm−1) at 1508±2 cm⁻¹, 1609±2 cm⁻¹ and 1631±2 cm⁻¹.

In another embodiment, the present invention provides a crystalline Form 2 of Compound 1 channel hydrate mono hydrochloride, wherein said crystalline Form 2 has a Raman spectrum comprising Raman shift peaks (cm−1) at 1508±2 cm⁻¹, 1609±2 cm⁻¹ and 1631±2 cm⁻¹, 864±2 cm^{-l}and 786±2 cm⁻¹.

In another embodiment, the present invention provides a crystalline Form 2 of Compound 1 channel hydrate mono hydrochloride, wherein said crystalline Form 2 has a solid state NMR spectrum comprising ¹³C chemical shifts at 165.9±0.2, 53.3±0.2 and 23.2±0.2 ppm.

In another embodiment, the present invention provides a crystalline Form 2 of Compound 1 channel hydrate mono hydrochloride, wherein said crystalline Form 2 has a solid state NMR spectrum comprising ¹³C chemical shifts at 165.9±0.2, 53.3±0.2 and 23.2±0.2, 115.2±0.2 and 156.6±0.2 ppm.

In another embodiment, the present invention provides a crystalline Form 2 of Compound 1 channel hydrate mono hydrochloride, wherein said crystalline Form 2 has a solid state NMR spectrum comprising an ¹⁹F chemical shift at −118.5±0.2 ppm.

In another embodiment, the present invention provides a crystalline Form 2 of Compound 1 channel hydrate mono hydrochloride, wherein said crystalline Form 2 has a powder X-ray diffraction pattern comprising peaks at positions essentially the same as shown in FIG. 5.

In another embodiment, the present invention provides a crystalline Form 2 of Compound 1 channel hydrate mono hydrochloride, wherein said crystalline Form 2 has a Raman spectrum comprising Raman shift peaks (cm−1) at positions essentially the same as shown in FIG. 8.

In another embodiment, the present invention provides a crystalline Form 2 of Compound 1 channel hydrate mono hydrochloride, wherein said crystalline Form 2 has a solid state NMR spectrum comprising ¹³C chemical shifts at positions essentially the same as shown in FIG. 6.

In another embodiment, the present invention provides a crystalline Form 2 of Compound 1 channel hydrate mono hydrochloride, wherein said crystalline Form 2 has a solid state NMR spectrum comprising an ¹⁹F chemical shift at position(s) essentially the same as shown in FIG. 7.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2, and a Raman spectrum comprising Raman shift peaks (cm−1) at at least one of 1508±2 cm⁻¹, 1609±2 cm^{-l}and 1631±2 cm⁻¹.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2, and a Raman spectrum comprising Raman shift peaks (cm−1) at positions essentially the same as shown in FIG. 8.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2, and a solid state NMR spectrum comprising ¹³C chemical shifts at at least one of 165.9±0.2, 53.3±0.2 and 23.2±0.2 ppm.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2, and a solid state NMR spectrum comprising ¹³C chemical shifts at positions essentially the same as shown in FIG. 6.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2, and a solid state NMR spectrum comprising an ¹⁹F chemical shift at −118.5±0.2 ppm.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2, and a solid state NMR spectrum comprising an ¹⁹F chemical shift at position(s) essentially the same as shown in FIG. 7.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of at least one of 7.2±0.2, 15.7±0.2 and 18.9±0.2, and a solid state NMR spectrum comprising an ¹⁹F chemical shift at −118.5±0.2 ppm.

In another embodiment, the present invention provides a crystalline Form 2 of Compound 1 channel hydrate mono hydrochloride, wherein said crystalline Form 2 has a

Raman spectrum comprising Raman shift peaks (cm−1) at at least one of 1508±2 cm⁻¹, 1609±2 cm⁻¹ and 1631±2 cm⁻¹, and a solid state NMR spectrum comprising an ¹⁹F chemical shift at −118.5 35 0.2 ppm.

In another embodiment, the present invention provides a crystalline Form 2 of Compound 1 channel hydrate mono hydrochloride, wherein said crystalline Form 2 has a solid state NMR spectrum comprising ¹³C chemical shifts at at least one of 165.9±0.2, 53.3±0.2 and 23.2±0.2 ppm, and a solid state NMR spectrum comprising an ¹⁹F chemical shift at −118.5±0.2 ppm.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2, a Raman spectrum comprising Raman shift peaks (cm−1) at at least one of 1508±2 cm⁻¹, 1609±2 cm⁻¹ and 1631±2 cm⁻¹, and a solid state NMR spectrum comprising ¹³C chemical shifts at at least one of 165.9±0.2, 53.3±0.2 and 23.2±0.2 ppm.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2, a Raman spectrum comprising Raman shift peaks (cm—1) at at least one of 1508±2 cm⁻¹, 1609±2 cm⁻¹ and 1631±2 cm⁻¹, and a solid state NMR spectrum comprising an ¹⁹F chemical shift at −118.5±0.2 ppm.

In another embodiment, the present invention provides a crystalline Form 2 of

Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2, a solid state NMR spectrum comprising ¹³C chemical shifts at at least one of 165.9±0.2, 53.3±0.2 and 23.2±0.2 ppm, and a solid state NMR spectrum comprising an ¹⁹F chemical shift at −118.5±0.2 ppm.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of at least one of 7.2±0.2, 15.7±0.2 and 18.9±0.2, a Raman spectrum comprising Raman shift peaks (cm−1) at at least one of 1508±2 cm⁻¹, 1609±2 cm⁻¹ and 1631±2 cm⁻¹, and a solid state NMR spectrum comprising an ¹⁹F chemical shift at −118.5±0.2 ppm.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of at least one of 7.2±0.2, 15.7±0.2 and 18.9±0.2, a solid state NMR spectrum comprising ¹³C chemical shifts at at least one of 165.9±0.2, 53.3±0.2 and 23.2±0.2 ppm, and a solid state NMR spectrum comprising an ¹⁹F chemical shift at −118.5±0.2 ppm.

In another embodiment, the present invention provides a crystalline Form 2 of Compound 1 channel hydrate mono hydrochloride, wherein said crystalline Form 2 has a Raman spectrum comprising Raman shift peaks (cm−1) at at least one of 1508±2 cm⁻¹, 1609 ±2 cm⁻¹ and 1631±2 cm⁻¹, a solid state NMR spectrum comprising ¹³C chemical shifts at at least one of 165.9±0.2, 53.3±0.2 and 23.2±0.2 ppm, and a solid state NMR spectrum comprising an ¹⁹F chemical shift at −118.5±0.2 ppm.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of 7.2±0.2, 15.7±0.2 and 18.9±0.2, a Raman spectrum comprising Raman shift peaks (cm−1) at at least one of 1508±2 cm⁻¹, 1609±2 cm⁻¹ and 1631±2 cm⁻¹, a solid state NMR spectrum comprising ¹³C chemical shifts at at least one of 165.9±0.2, 53.3±0.2 and 23.2±0.2 ppm, and a solid state NMR spectrum comprising an ¹⁹F chemical shift at −118.5±0.2 ppm.

In another embodiment, the present invention provides a crystalline Form 2 of Compound 1 channel hydrate mono hydrochloride, wherein said crystalline Form 2 has a powder X-ray diffraction pattern comprising peaks at diffraction angle (2 degrees θ) of at least one of 7.2±0.2, 15.7±0.2 and 18.9±0.2, a Raman spectrum comprising Raman shift peaks (cm−1) at at least one of 1508±2 cm⁻¹, 1609±2 cm⁻¹ and 1631±2 cm⁻¹, a solid state NMR spectrum comprising ¹³C chemical shifts at at least one of 165.9±0.2, 53.3±0.2 and 23.2±0.2 ppm, and a solid state NMR spectrum comprising an ¹⁹F chemical shift at −118.5±0.2 ppm.

A further aspect of the present invention provides a pharmaceutical composition comprising any of the crystalline forms of Compound 1 as described herein. In a further aspect, the invention provides an oral dosage form comprising any of the crystalline forms of Compound 1 or pharmaceutical compositions described herein. For example, in one embodiment the oral dosage form is a tablet, pill, dragee core, or capsule. For example, in one embodiment, the oral dosage form is a tablet or capsule. Further, for example, in one embodiment the invention provides a tablet comprising any of the crystalline forms of Compound 1 or pharmaceutical compositions described herein. For example, in one embodiment the tablet comprises from about 5mg to about 10 mg, from about 10mg to about 20 mg, from about 20mg to about 30 mg, from about 30mg to about 40 mg, from about 40mg to about 50 mg, from about 50mg to about 75 mg, from about 75mg to about 100 mg, from about 100mg to about 150 mg, from about 150mg to about 200 mg, from about 200mg to about 300 mg, from about 300mg to about 400 mg, or from about 400mg to about 500 mg, of a crystalline form of Compound 1. Tablets of other doses are also possible. In a further embodiment, the crystalline form of Compound 1 is Form 1. In a still further embodiment, the crystalline form of Compound 1 is Form 2. In a still further embodiment, the present invention provides a pharmaceutical composition comprising Form 1 and Form 2 together, including mixtures thereof.

A further aspect of the present invention provides a method of treating cancer in a mammal, the method comprising administering to the mammal a therapeutically effective amount of any of the crystalline forms of Compound 1 or any of the pharmaceutical compositions described herein.

In certain embodiments of the invention clinical doses of Compound (Form 1 or Form 2) include 20 mg, 40 mg, 80 mg, 150 mg, 250 mg, 500 mg, 625 mg administered once or twice daily. In certain embodiments such administration is in combination with palbociclib (or other CDK inhibitor), or in combination with palbociclib (or other CDK inhibitor) plus letrozole. In certain other embodiments such administration is in combination with enzalutamide.

In a particular aspect of any of the preceding method embodiments, the method further comprises administering one or more anti-tumor agents, anti-angiogenesis agents, signal transduction inhibitors, or antiproliferative agents.

Embodiments of the invention further include combinations of Form 1 of Compound 1 plus a second therapeutic agent (including a therapeutic agent selected from anti-tumor agents, anti-angiogenesis agents, signal transduction inhibitors, or antiproliferative agents).

Embodiments of the invention further include combinations of Form 2 of Compound 1 plus a second therapeutic agent (including a therapeutic agent selected from anti-tumor agents, anti-angiogenesis agents, signal transduction inhibitors, or antiproliferative agents).

### Definitions

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of “treating” as defined immediately above.

As used herein, the term “Compound 1” means the chemical compound 4-(2-(5-chloro-2-fluorophenyl) -5-isopropylpyridin-4-ylamino)-N-(1,3-dihydroxypropan-2-yl)nicotinamide, also represented by the structural formula:

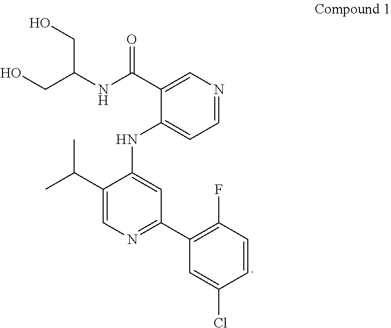 [SMILES: CC(C)c1cnc(-c2cc(Cl)ccc2F)cc1Nc1ccncc1C(=O)NC(CO)CO]

As used herein, the term “substantially pure” with reference to a particular crystalline or amorphous form means that the crystalline or amorphous form includes less than 10%, preferably less than 5%, preferably less than 3%, preferably less than 1% by weight of any other physical forms of the compound.

As used herein, the term “essentially the same” with reference to X-ray diffraction peak positions means that typical peak position and intensity variability are taken into account. For example, one skilled in the art will appreciate that the peak positions (20) will show some variability, typically as much as 0.1 to 0.2 degrees, depending on the solvents being used, as well as on the apparatus being used to measure the diffraction. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only. Similarly, as used herein, “essentially the same” with reference to solid state NMR spectra and Raman spectra is intended to also encompass the variabilities associated with these analytical techniques, which are known to those of skill in the art. For example, ¹³C chemical shifts measured in solid state NMR will typically have a variability of up to 0.2 ppm for well defined peaks, and even larger for broad lines, while Raman shifts will typically have a variability of about 2 cm⁻¹.

The term “polymorph” refers to different crystalline forms of the same compound and includes, but is not limited to, other solid state molecular crystalline forms including hydrates (e.g., bound water present in the crystalline structure) and solvates (e.g., bound solvents other than water) of the same compound.

The term “2 theta value” or “2θ” refers to the peak position in degrees based on the experimental setup of the X-ray diffraction experiment and is a common abscissa unit in diffraction patterns. The experimental setup requires that if a reflection is diffracted when the incoming beam forms an angle theta (θ) with a certain lattice plane, the reflected beam is recorded at an angle 2 theta (2θ). It should be understood that reference herein to specific 2θ values for a specific polymorphic form is intended to mean the 2θ values (in degrees) as measured using the X-ray diffraction experimental conditions as described herein. For example, as described herein, CuKα (wavelength 1.54056 Å) was used as the source of radiation.

The term “amorphous” refers to any solid substance which (i) lacks order in three dimensions, or (ii) exhibits order in less than three dimensions, order only over short distances (e.g., less than 10 Å), or both. Thus, amorphous substances include partially crystalline materials and crystalline mesophases with, e.g. one- or two-dimensional translational order (liquid crystals), orientational disorder (orientationally disordered crystals), or conformational disorder (conformationally disordered crystals). Amorphous solids may be characterized by known techniques, including X-ray powder diffraction (XRPD) crystallography, solid state nuclear magnet resonance (ssNMR) spectroscopy, differential scanning calorimetry (DSC), or some combination of these techniques. As illustrated, below, amorphous solids give diffuse XRPD patterns, typically comprised of one or two broad peaks (i.e., peaks having base widths of about 5° 2θ or greater).

The term “channel hydrate” refers to hydrate structures with open structural voids where the water molecules may fully or partly escape through the channels (voids) without siginificant changes in the crystal structure. See: Braun, D. E., Griesser, U. J., Cryst. Growth Des. 2016, 16, 6111-6121.

The term “crystalline” refers to any solid substance exhibiting three-dimensional order, which in contrast to an amorphous solid substance, gives a distinctive XRPD pattern with sharply defined peaks.

The term “solvate” describes a molecular complex comprising the drug substance and a stoichiometric or non-stoichiometric amount of one or more solvent molecules (e.g., ethanol). When the solvent is tightly bound to the drug the resulting complex will have a well-defined stoichiometry that is independent of humidity. When, however, the solvent is weakly bound, as in channel solvates and hygroscopic compounds, the solvent content will be dependent on humidity and drying conditions. In such cases, the complex will often be non-stoichiometric.

The term “hydrate” describes a solvate comprising the drug substance and a stoichiometric or non-stoichiometric amount of water.

The term “powder X-ray diffraction pattern” or “PXRD pattern” refers to the experimentally observed diffractogram or parameters derived therefrom. Powder X-Ray diffraction patterns are characterized by peak position (abscissa) and peak intensities (ordinate).

The term “pharmaceutical composition” refers to a composition comprising one or more of the polymorphic forms of Compound 1 described herein, and other chemical components, such as physiologically/pharmaceutically acceptable carriers, diluents, vehicles and/or excipients. The purpose of a pharmaceutical composition is to facilitate administration of a compound to an organism, such as a human or other mammal.

The term “pharmaceutically acceptable” “carrier”, “diluent”, “vehicle”, or “excipient” refers to a material (or materials) that may be included with a particular pharmaceutical agent to form a pharmaceutical composition, and may be solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

### BRIEF DESCRIPTION OF THE DRAWINGS

FIG. 1 shows a PXRD pattern of Compound 1, Form 1 anhydrous mono hydrochloride salt.

FIG. 2 shows a ¹³C solid state NMR spectrum of Compound 1, Form 1 anhydrous mono hydrochloride salt. Spinning side bands are indicated with hashed marks.

FIG. 3 shows a ¹⁹F solid state NMR spectrum of Compound 1, Form 1 anhydrous mono hydrochloride salt. Spinning side bands are indicated with hashed marks.

FIG. 4 shows a Raman spectrum of Compound 1, Form 1 anhydrous mono hydrochloride salt.

FIG. 5 shows a PXRD pattern of Compound 1, Form 2 channel hydrate mono hydrochloride salt.

FIG. 6 shows a ¹³C solid state NMR spectrum of Compound 1, Form 2 channel hydrate mono hydrochloride salt. Spinning side bands are indicated with hashed marks.

FIG. 7 shows ¹⁹F solid state NMR spectrum of Compound 1, Form 2 channel hydrate mono hydrochloride salt. Spinning side bands are indicated with hashed marks.

FIG. 8 shows a Raman spectrum of Compound 1, Form 2 channel hydrate mono hydrochloride salt.

FIG. 9 shows an asymmetric unit of Compound 1, Form 1 with displacement parameters drawn at 50% probability. The asymmetric unit is comprised of one molecule of Compound 1 protonated and one molecule of chorine deprotonated.

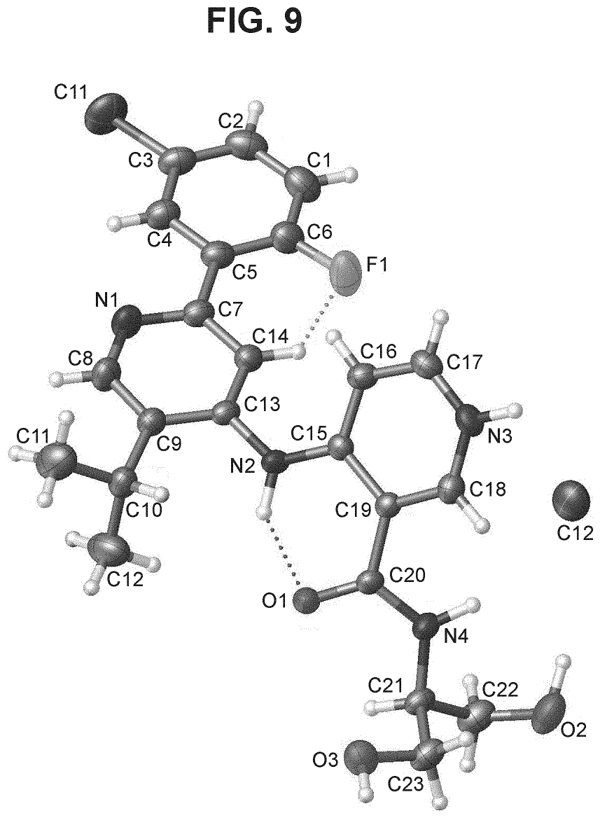

### DETAILED DESCRIPTION OF THE INVENTION

It has been found that Compound 1 can exist in multiple crystalline forms (polymorphs). These forms may be used in a formulated product for the treatment of hyperproliferative indications, including cancer. Each form may have advantages over the others in terms of properties such as bioavailability, stability, and manufacturability. Novel crystalline forms of Compound 1 have been discovered which are likely to be more suitable for bulk preparation and handling than other polymorphic forms. Processes for producing polymorphic forms of Compound 1 in high purity are described herein. Another object of the present invention is to provide a process for the preparation of each polymorphic form of Compound 1, substantially free from other polymorphic forms of Compound 1. Additionally it is an object of the present invention to provide pharmaceutical formulations comprising Compound 1 in different polymorphic forms as discussed above, and methods of treating hyperproliferative conditions by administering such pharmaceutical formulations.

Each crystalline form of Compound 1 can be characterized by one or more of the following: powder X-ray diffraction pattern (i.e., X-ray diffraction peaks at various diffraction angles (2)), solid state nuclear magnetic resonance (NMR) spectral pattern, Raman spectral diagram pattern, aqueous solubility, light stability under International Conference on Harmonization (ICH) high intensity light conditions, and physical and chemical storage stability. For example, polymorphic Forms 1 and 2 of Compound 1 were each characterized by the positions and relative intensities of peaks in their powder X-ray diffraction patterns. The powder X-ray diffraction parameters differ for each of the polymorphic forms of Compound 1. For example, Forms 1 and 2 of Compound 1 can therefore be distinguished from each other and from other polymorphic forms of Compound 1 by using powder X-ray diffraction. To perform an X-ray diffraction measurement on an instrument as described herein the sample is typically placed into a holder which has a cavity. The sample powder is pressed by a glass slide or equivalent to ensure a random surface and proper sample height. The sample holder is then placed into the instrument. The incident X-ray beam is directed at the sample, initially at a small angle relative to the plane of the holder, and then moved through an arc that continuously increases the angle between the incident beam and the plane of the holder. Measurement differences associated with such X-ray powder analyses result from a variety of factors including: (a) errors in sample preparation (e.g., sample height); (b) instrument errors (e.g., flat sample errors); (c) calibration errors; (d) operator errors (including those errors present when determining the peak locations); and (e) the nature of the material (e.g., preferred orientation and transparency errors). Calibration errors and sample height errors often result in a shift of all the peaks in the same direction. Small differences in sample height when using a flat holder will lead to large displacements in PXRD peak positions. These shifts can be identified from the X-ray diffractogram and can be eliminated by compensating for the shift (applying a systematic correction factor to all peak position values) or recalibrating the instrument. As mentioned above, it is possible to rectify measurements from the various machines by applying a systematic correction factor to bring the peak positions into agreement. In general, this correction factor will bring the measured peak positions from the PXRD instrument (typically made by Bruker) into agreement with the expected peak positions and may be in the range of 0 to 0.2 degrees (2θ). One of skill in the art will appreciate that the peak positions (2θ) will show some inter-apparatus variability, typically as much as 0.1 to 0.2 degrees (2θ). Accordingly, where peak positions (2θ) are reported, one of skill in the art will recognize that such numbers are intended to encompass such inter-apparatus variability. Furthermore, where the crystalline forms of the present invention are described as having a powder X-ray diffraction pattern essentially the same as that shown in a given figure, the term “essentially the same” is also intended to encompass such inter-apparatus variability in diffraction peak positions. Further, one skilled in the art will appreciate that relative peak intensities will show inter-apparatus variability as well as variability due to the degree of crystallinity, preferred orientation, prepared sample surface, and other factors known to those skilled in the art, and should be taken as qualitative measures only.

The different crystalline forms of the present invention can also be characterized using solid state NMR spectroscopy. ¹³C solid state spectra and ¹⁹F solid state spectra can be collected as described herein.

The different crystalline forms of the present invention can also be characterized using Raman spectroscopy. Raman spectra can be collected as described herein.

The solid forms of the present invention may also comprise more than one polymorphic form. One of skill in the art will also recognize that crystalline forms of a given compound can exist in substantially pure forms of a single polymorph, but can also exist in a crystalline form that comprises two or more different polymorphs or amorphous forms. Where a solid form comprises two or more polymorphs, the X-ray diffraction pattern will have peaks characteristic of each of the individual polymorphs of the present invention. For example, a solid form that comprises two polymorphs will have a powder X-ray diffraction pattern that is a convolution of the two X-ray diffraction patterns that correspond to the substantially pure polymorphic forms. For example, a solid form of Compound 1 can contain a first and second polymorphic form where the solid form contains at least 10% by weight of the first polymorph. In a further example, the solid form contains at least 20% by weight of the first polymorph. Even further examples contain at least 30%, at least 40%, or at least 50% by weight of the fist polymorph. One of skill in the art will recognize that many such combinations of several individual polymorphs and amorphous forms in varying amounts are possible.

The active agents (i.e., the polymorphs, or solid forms comprising two or more such polymorphs, of Compound 1 described herein) of the invention may be formulated into pharmaceutical compositions suitable for mammalian medical use. Any suitable route of administration may be employed for providing a patient with an effective dosage of any of the polymorphic forms of Compound 1. For example, peroral or parenteral formulations and the like may be employed. Dosage forms include capsules, tablets, dispersions, suspensions and the like, e.g. enteric-coated capsules and/or tablets, capsules and/or tablets containing enteric-coated pellets of Compound 1. In all dosage forms, polymorphic forms of Compound 1 can be admixtured with other suitable constituents. The compositions may be conveniently presented in unit dosage forms, and prepared by any methods known in the pharmaceutical arts. Pharmaceutical compositions of the invention comprise a therapeutically effective amount of the active agent and one or more inert, pharmaceutically acceptable carriers, and optionally any other therapeutic ingredients, stabilizers, or the like. The carrier(s) must be pharmaceutically acceptable in the sense of being compatible with the other ingredients of the formulation and not unduly deleterious to the recipient thereof. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80”, and pluronics such as F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in Remington: The Science & Practice of Pharmacy, 19^{th} ed., Williams & Williams, (1995), and in the “Physician's Desk Reference”, 52^{nd} ed., Medical Economics, Montvale, N.J. (1998), and in Handbook of Pharmaceutical Excipients, 3^{rd}. Ed., Ed. A. H. Kibbe, Pharmaceutical Press, 2000. The active agents of the invention may be formulated in compositions including those suitable for oral, rectal, topical, nasal, ophthalmic, or parenteral (including intraperitoneal, intravenous, subcutaneous, or intramuscular injection) administration.

The amount of the active agent in the formulation will vary depending upon a variety of factors, including dosage form, the condition to be treated, target patient population, and other considerations, and will generally be readily determined by one skilled in the art. A therapeutically effective amount will be an amount necessary to modulate, regulate, or inhibit a protein kinase. In practice, this will vary widely depending upon the particular active agent, the severity of the condition to be treated, the patient population, the stability of the formulation, and the like. Compositions will generally contain anywhere from about 0.001% by weight to about 99% by weight active agent, preferably from about 0.01% to about 5% by weight active agent, and more preferably from about 0.01% to 2% by weight active agent, and will also depend upon the relative amounts of excipients/additives contained in the composition.

A pharmaceutical composition of the invention is administered in conventional dosage form prepared by combining a therapeutically effective amount of an active agent as an active ingredient with one or more appropriate pharmaceutical carriers according to conventional procedures. These procedures may involve mixing, granulating and compressing or dissolving the ingredients as appropriate to the desired preparation.

The pharmaceutical carrier(s) employed may be either solid or liquid. Exemplary solid carriers include lactose, sucrose, talc, gelatin, agar, pectin, acacia, magnesium stearate, stearic acid and the like. Exemplary liquid carriers include syrup, peanut oil, olive oil, water and the like. Similarly, the carrier(s) may include time-delay or time-release materials known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation can be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an active agent can be dissolved in an aqueous solution of an organic or inorganic acid, such as 0.3 M solution of succinic acid or citric acid. If a soluble salt form is not available, the active agent may be dissolved in a suitable co-solvent or combinations of co-solvents. Examples of suitable co-solvents include, but are not limited to, alcohol, propylene glycol, polyethylene glycol 300, polysorbate 80, gylcerin and the like in concentrations ranging from 0-60% of the total volume. The composition may also be in the form of a solution of a salt form of the active agent in an appropriate aqueous vehicle such as water or isotonic saline or dextrose solution.

It will be appreciated that the actual dosages of Compound 1 used in the compositions of this invention will vary according to the particular polymorphic form being used, the particular composition formulated, the mode of administration and the particular site, host and disease being treated. Those skilled in the art using conventional dosage-determination tests in view of the experimental data for an agent can ascertain optimal dosages for a given set of conditions. For oral administration, an exemplary daily dose generally employed is from about 0.001 to about 1000 mg/kg of body weight, more preferably from about 0.001 to about 50 mg/kg body weight, with courses of treatment repeated at appropriate intervals. Administration of prodrugs is typically dosed at weight levels that are chemically equivalent to the weight levels of the fully active form. In the practice of the invention, the most suitable route of administration as well as the magnitude of a therapeutic dose will depend on the nature and severity of the disease to be treated. The dose, and dose frequency, may also vary according to the age, body weight, and response of the individual patient. In general, a suitable oral dosage form may cover a dose range from 0.5 mg to 100 mg of active ingredient total daily dose, administered in one single dose or equally divided doses. A preferred amount of Compound 1 in such formulations is from about 0.5 mg to about 20 mg, such as from about 1 mg to about 10 mg or from about 1 mg to about 5 mg.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations that can be used pharmaceutically.

For oral administration, a polymorphic form of Compound 1 can be formulated readily by combining the active agent with pharmaceutically acceptable carriers known in the art. Such carriers enable the compounds of the invention to be formulated as tablets, pills, dragees, capsules, gels, syrups, slurries, suspensions and the like, for oral ingestion by a patient to be treated. Pharmaceutical preparations for oral use can be obtained using a solid excipient in admixture with the active agent, optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/cilary, lens, choroid/retina and selera. The pharmaceutically acceptable ophthalmic vehicle may be, for example, an ointment, vegetable oil, or an encapsulating material. An active agent of the invention may also be injected directly into the vitreous and aqueous humor or subtenon.

Alternatively, the active ingredient may be in powder form for constitution with a suitable vehicle, e.g., sterile pyrogen-free water, before use. The compounds may also be formulated in rectal or vaginal compositions such as suppositories or retention enemas, e.g., containing conventional suppository bases such as cocoa butter or other glycerides.

In addition to the formulations described above, the polymorphic forms may also be formulated as a depot preparation. Such long-acting formulations may be administered by implantation (for example, subcutaneously or intramuscularly) or by intramuscular injection. Thus, for example, the polymorphic forms may be formulated with suitable polymeric or hydrophobic materials (for example, as an emulsion in an acceptable oil) or ion-exchange resins, or as sparingly soluble derivatives, for example, as a sparingly soluble salt.

Additionally, polymorphic forms of Compound 1 may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compound for a few weeks up to over 100 days.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Polymorphic forms of Compound 1 are useful for mediating the activity of protein kinases. More particularly, the polymorphic forms are useful as anti-angiogenesis agents and as agents for modulating and/or inhibiting the activity of protein kinases, such as the activity associated with VEGF, FGF, CDK complexes, TEK, CHK1, LCK, FAK, and phosphorylase kinase among others, thus providing treatments for cancer or other diseases associated with cellular proliferation mediated by protein kinases in mammals, including humans.

Therapeutically effective amounts of the herein-described Compound 1 polymorphs may be administered, typically in the form of a pharmaceutical composition, to treat diseases mediated by modulation or regulation of protein kinases. An “effective amount” is intended to mean that amount of an agent that, when administered to a mammal in need of such treatment, is sufficient to effect treatment for a disease mediated by the activity of one or more protein kinases, such as tyrosine kinases. Thus, a therapeutically effective amount of Compound 1 is a quantity sufficient to modulate, regulate, or inhibit the activity of one or more protein kinases such that a disease condition that is mediated by that activity is reduced or alleviated. “Treating” is intended to mean at least the mitigation of a disease condition in a mammal, such as a human, that is affected, at least in part, by the activity of one or more protein kinases, such as tyrosine kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition. Exemplary disease conditions include diabetic retinopathy, neovascular glaucoma, rheumatoid arthritis, psoriasis, age-related macular degeneration (AMD), and abnormal cell growth, such as cancer.

In one embodiment of this method, the abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In one embodiment of this method, the abnormal cell growth is prostate cancer.

In one embodiment of this method, the abnormal cell growth is breast cancer.

In one embodiment of the present invention the cancer is lung cancer (NSCLC and SCLC), cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non hodgkins's lymphoma, or spinal axis tumors, or a combination of one or more of the foregoing cancers.

In a particular embodiment, the cancer is cancer of the thyroid gland, cancer of the parathyroid gland, pancreatic cancer, colon cancer, or renal cell carcinoma.

In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a polymorphic form of Compound 1 that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

### EXAMPLES

The examples which follow will further illustrate the preparation of the distinct polymorphic forms of the invention, i.e. polymorphic Forms 1 and 2 of Compound 1, but are not intended to limit the scope of the invention as defined herein or as claimed below.

### Example 1A: Preparation of Compound 1 Anhydrous Mono Hydrochloride Form 1 Crystalline Polymorph

A 100-L, drop-bottom, glass jacketed reactor was equipped with a thermal control unit, and a condenser. A nitrogen bleed was applied and the temperature was set to 20° C. The reactor was charged with purified water (8.3 L, 2.5 vol) followed by a portion-wise addition of potassium phosphate tribasic (10.106 kg, 3 equiv) over 155 min to maintain the batch temperature at <35° C. 1,4-Dioxane (17.760 L, 5 vol) was added to the reactor and the biphasic mixture was purged 5 times by evacuating to 15″ Hg, holding for at least 2 min then releasing back to neutral pressure with nitrogen. 5-bromo-2-chloropyridin-4-amine (394 g, 1 equiv total), tricyclohexylphosphine (335.2 g, 0.075 equiv), and tris(dibenzylideneacetone)-dipalladium (364.0 g, 0.025 equiv) were charged to the reactor which was heated to 90±5° C. A 50-L, drop-bottom, glass jacketed reactor was equipped with a thermal control unit, and a condenser. A nitrogen bleed was applied and the reactor was charged with purified water (8.3 L, 2.5 vol), 1,4-dioxane (17 L, 5 vol), and trifluoro(prop-1-en-2-yl)borate (2590 g, 1.10 equiv). The resulting solution was purged 5 times by evacuating to 15″ Hg, holding for at least 2 min then releasing back to neutral pressure with nitorogen. The solution was transferred to the 100-L reactor over 1 h and 8 min while maintaining the temperature at 80-95° C. The contents of the 100-L reactor were stirred for 1 h at 90±5° C. The batch was then cooled to 50±5° C. and phases were allowed to separate. Analysis revealed 0.4% 5-bromo-2-chloropyridin-4-amine, and the presence of 2-chloro-5-(prop-1-en-2-yl)pyridin-4-amine. The reaction was cooled to 20±5° C. and the aqueous phase was separated. The organic layer was diluted with toluene (17 L, 5 vol) then washed with purified water (17 L, 5 vol). The aqueous layer was extracted with toluene (17 L, 2×5 vol). The combined organic layer was washed with purified water (17 L, 2×5 vol) then filtered and placed on top of cellulose filter paper). The reactor was rinsed with toluene (7 L, 2 vol) and the rinse was transferred to the filter funnel. The filtrate was returned to the reactor via an in-line filter and was concentrated to 17 L (5 vol) under reduced pressure at ≤60° C. The vacuum was released to neutral pressure using nitrogen, temperature was adjusted to <30° C., and toluene (33 L, 10 vol) was charged to the reactor. The concentration operation was repeated at ≤60° C. The temperature was adjusted to 20±5° C.

THF (7 L, 2 vol) was charged to the batch followed by 4-chloro-N-(2,2-dimethyl-1,3-dioxan-5-yl)nicotinamide (2907 g, 0.67 equiv). The solution was cooled to 5±5 ° C. before 1 molar lithium bis(trimethylsilyl)amide (LiHMDS) in THF solution (28.52 kg, 3.00 equiv) was added over 1 h and 35 min while maintaining the batch temperature <35° C., forming a slurry. Temperature was adjusted to 60±5° C. and the reaction was stirred for 16 h. Temperature was adjusted to 5±5° C. and purified water (29 L, 10 vol) was charged to the reactor over 50 min while maintaining the temperature at <35° C. Temperature was adjusted to 20±5° C. and the phases were separated. The aqueous layer was extracted with 2-MeTHF (14 L, 5 vol). The combined organic layer was returned to the reactor and distilled under vacuum to 14 L (5 vol) while maintaining the temperature at ≤60° C. Isopropyl acetate (iPrOAc, 43 L, 15 vol) was charged to the reactor and the reaction was distilled to 26 L (9 vol) while at ≤60° C. Additional iPrOAc (3 L, 1 vol) was charged to the reactor. Temperature was adjusted to 55±5° C. and stirred at that temperature for 1 h. Temperature was then adjusted to 20±5° C. over 2 h and 21 min and stirred overnight. The reaction was filtered, washed with iPrOAc (2×6 L, 2 vol), and conditioned under nitrogen for 1 h. The filter cake was dried to constant weight at 45±5° C. under vacuum to afford 3031 g 4-(2-chloro-5-(prop-1-en-2-yl) pyridin-4-ylamino)-N-(2,2-dimethyl-1,3-dioxan-5-yl)nicotinamide as a tan solid.

Methanol (38 mL, 9.5 L/Kg) and triethylamine (2.95 g, 0.029 mol, 3.0 equiv) were added to a 100 mL reactor and heated to 20° C. 4-(2-chloro-5-(prop-1-en-2-yl)pyridin-4-ylamino)-N-(2,2-dimethyl-1,3-dioxan-5-yl) nicotinamide [617] (3.99 g, 0.010 mol, 1.0 eq) was added to the reactor and a yellow slurry was obtained. 2-dicyclohexylphosphino-22 ,6′-dimethoxybiphenyl (SPhos) (42 mg, 0.102 mmol, 0.01 equiv) was added to the reactor. 5-chloro-2-fluorophenylboronic acid (1.89 g, 0.011 mol, 1.1 equiv) was added to the reactor. The resulting slurry was sparged with nitrogen sub-surface for 5 minutes. Palladium (II) acetate (22 mg, 0.098 mmol, 0.01 equiv) was added to the reactor. Solids were rinsed into the reactor with methanol (2 mL, 0.5 L/Kg). The slurry was sparged with nitrogen sub-surface for 6 minutes, heated to 65±5° C., and heated at 60 to 70° C. for 5 to 6 hours. A solid product precipitates after <1 hour at 60 to 70° C. A solution of N-acetyl-L-cysteine (0.80 g, 0.005 mol, 0.2 Kg/Kg) in methanol (6 mL, 1.5 L/Kg) was prepared in a second vessel and triethylamine (1.6 mL, 0.011 mol, 0.4 L/Kg) was added. A clear colorless solution (2 L/Kg) was obtained. Temperature was reduced to 60° C. The N-acetyl-L-cysteine solution was added to the reaction mixture over 30 minutes maintaining a temperature of 55 to 65° C. Temperature was increased to 70° C. at 1° C./min, and then held at 60 to 70° C. for 1 hour to afford a cream slurry. The reaction mixture was cooled to 18 to 22° C. at a rate of 0.2° C./min and stirred for 16 hours at 20° C. The mixture was then filtered under 400 mbar pressure and the filter cake deliquored to afford a filtrate that was clear orange (34 mL). Methanol (36 mL, 9 L/Kg) was added to the vessel and the contents were adjusted to 18 to 22° C. In each of three washes a portion of the vessel contents (12 mL, 3 L/Kg) was used to wash the filter cake. The cake volume was 9.1 mL. The filter cake was dried on the filter for 15 minutes and then dried under vacuum at 60° C. for 12 hours and determined to be 4-(2-(5-chloro-2-fluorophenyl)-5-(prop-1-en-2-yl)pyridin-4-ylamino)-N-(2,2-dimethyl-1,3-dioxan-5-yl) nicotinamide 4.04 g, 82%.

Tetrahydrofuran (116 mL, 12 mL/g), and then 4-(2-(5-chloro-2-fluorophenyl)-5-(prop-1-en-2-yl) pyridin-4-ylamino)-N-(2,2-dimethyl-1,3-dioxan-5-yl)nicotinamide (9.65 g, 1 wt), were charged to a reactor. 5% platinum on carbon (JM Type 103, 2.01 g) was added to the reactor, and the mixture was stirred and purged with nitrogen to 50-60 psi and then released to ambient pressure, repeated three times. The reaction was then purged with hydrogen to 50-60 psi and released to ambient pressure, repeated three times. The reaction mixture was heated to 50-55° C., maintained for 24 hours, and cooled to 20-25° C. The reaction mixture was purged with nitrogen to 50-60 psi and released to ambient pressure, repeated three times. The reaction mixture was filtered through a pad of filter aid (38.6 g,4 g/g) to remove the catalyst. The filter pad was washed through with tetrahydrofuran (48.3 mL, 5 mL/g). The reaction mixture was reduced to 48.3 mL. The reaction mixture was heated to 67° C. The tetrahydrofuran was distilled out and acetonitrile (48.3 mL,10 mL/g) was added, maintaining constant overall volume. 18. The distillation was continued until temperature reached 80° C. The mixture was cooled to 75° C., forming a slurry. The slurry was held at 75° C. for 2 hours, cooled to 20° C., and then held at 20° C. for 8.5 hours. Acetonitrile (24.2mL, 5 mL/g) was added to the slurry, which was then filtered. The cake was washed with acetonitrile (24.2 mL, 5 mL/g), slurried on the filter with acetonitrile (5 mL/g), and then washed with acetonitrile (5 mL/g). The product, 4-(2-(5-chloro-2-fluorophenyl)-5-isopropylpyridin-4-ylamino)-N-(2,2-dimethyl-1,3-dioxan-5-yl) nicotinamide was removed from the filter and dried in a vacuum oven at 60° C. for 72 hours. Yield: 3.85 g, 80%.

Methanol (25 mL, 10 mL/g) was added to a reaction vessel and heated to 20° C. 4-(2-(5-chloro -2-fluorophenyl)-5-isopropylpyridin-4-ylamino)-N-(2,2-dimethyl-1,3-dioxan-5-yl)nicotinamide (2.49 g, 4.99 mmol, 1 eq. LR) was then added to the vessel to obtain a slurry that was held at 20° C. HCl (0.52 g, 5.24 mmol, 1.05 eq.) was added to the vessel, which was stirred at 20° C. for 30 minutes. The reaction was then heated to 60° C. and held for 2 h. Quadrapure® TU (0.50 g, 0.2 g/g) was added and the reaction was held at 60° C. for 2 h. The reaction was filtered through Dicalite in order to remove the metal scavenger. The filter pad was washed with Methanol (7.5 mL, 3 mL/g).

In a separate vessel Metal Scavenger (Silicycle Si-Thiol?) (0.50 g, 0.2 g/g) was slurried in Methanol (2.5 mL, 1 mL/g), and then transferred to the first vessel followed by a Methanol (0.6 mL, 0.25 mL/g) line rinse. The mixture was held at 60° C. for 16 h, and then filtered through Dicalite in order to remove the scavenger. The filter pad was washed with methanol (7.5 mL, 3 mL/g) at 60° C. The reaction was distilled under atmospheric pressure down to around 5 mL/g. To the vessel was added Ethanol (25 mL, 10 mL/g) The reaction was distilled under atmospheric pressure down to around 5 mL/g. To the vessel was added ethanol (25 mL, 10 mL/g) to bring the total volume to 15 mL/g. The reaction was cooled to 20° C. at a rate of 0.5° C./min. Water was added to give a water content of 8.96%. The reaction was then heated to 60° C. and held for at least 1 h. The reaction was then cooled to 40° C. over at least 4 h (0.08° C./ min). The reaction was held at 40° C. for 8 h. The reaction was then cooled to 10 ° C. over 4 h (0.1° C./ min), and held at 10° C. for 12 h. The cake was washed with ethanol (5.0 mL, 2 mL/g). The product was dried under vacuum for at least 12 h at 50° C. and determined to be 4-(2-(5-chloro-2-fluorophenyl)-5-isopropylpyridin-4-ylamino)-N-(1,3-dihydroxypropan -2-yl)nicotinamide mono hydrochloride. Yield: 65%

### Example 1B: Preparation Compound 1 Anhydrous Mono Hydrochloride Form 1 Crystalline Polymorph from Compound 1 Free Base

8.67 g (18.9 mmol, 1.0 equiv.) of 4-(2-(5-chloro-2-fluorophenyl)-5-isopropylpyridin-4-ylamino) -N-(1,3-dihydroxypropan-2-yl)nicotinamide (free base, see preparation at WO2015/103355 at Example 22) and 130 mL anhydrous ethanol (15 mL/g, 2230 mmol, 103 g, 130 mL) was charged to a first vessel, which was then heated to 75° C. Water (0.5 mL/g, 241 mmol, 4.34 g, 4.34 mL) and hydrochloric acid (12.2 Mol/L) (1.00 equiv., 18.9 mmol, 1.84 g, 1.55 mL) were charged to a second vessel and heated to 75° C. The contents of the second vessel were added to the first vessel. The resulting solution was filtered using speck-free conditions into a speck-free vessel, and then cooled to 50° C. over a period of 60 minutes, forming a slurry. The slurry was held at 50° C. over a period of 60 minutes, then cooled to 20° C. over a period of 120 minutes, and then held at 20° C. for a period of 8 hours. The white slurry was filtered over a filter cake, which was washed with 2 ml/g (17.34 ml) ethanol. The solid product was dried in an oven at 45 ° C. for a period of 8 hours. 4-(2-(5-chloro-2-fluorophenyl)-5-isopropylpyridin-4-ylamino)-N-(1,3-dihydroxypropan-2-yl)nicotinamide mono-HCl Form 1 was isolated (8.45 g, 90.3% yield).

### Example 1C: Additional Preparation Compound 1 Anhydrous Mono Hydrochloride Form 1 Crystalline Polymorph

3.0 g (1.0 equiv.) of 4-(2-(5-chloro-2-fluorophenyl)-5-isopropylpyridin-4-ylamino)-N-(1,3-dihydroxypropan-2-yl) nicotinamide mono hydrochloride was charged to an 100 ml reactor, which was then charged with ethanol (14 mL/g (Actual), 33 g, 42 mL, 120 equiv.) and water (1.1 mL/g (Actual), 3.3 g, 3.3 mL, 30 equiv.) to form a slurry. The slurry was heated to 75° C. to form a solution, and then cooled to 65° C. Additional 4-(2-(5-chloro-2-fluorophenyl)-5-isopropylpyridin -4-ylamino)-N-(1,3-dihydroxypropan-2-yl)nicotinamide mono hydrochloride (0.04 equiv. (Actual), 0.12 g, 0.040 equiv.) was charged to the reactor until the solution became cloudy. The reaction was cooled to 60° C. and held for one hour, then gradually cooled to 40° C. over the course of 4 hours. The reaction was held for 8 hours, then gradually cooled to 10° C. over the course of 8 hours. The reaction was held at 10° C. for 12 hours, then filtered over filter paper, washed with ethanol and dried under vacuum. The resulting solid Form 1 was isolated in an amber bottle (2.57 g (Actual), 2.57 g, 0.86 equiv.) Yield: 65%.

### Example 2A: Preparation of Compound 1 Channel Hydrate Mono Hydrochloride Form 2 Crystalline Polymorph

Solid Compound 1, Form 1 (for instance prepared as described in Examples 1A-1C) (0.3728 g) was added to a vial with a stirbar. Water (3.00 mL) was added and the mixture was stirred at room temperature. After stirring overnight, the solid was collected with vacuum filtration and dried on the benchtop. 399.6 mg (91% yield) of Form 2 was recovered.

### Example 2B: Preparation of Compound 1 Channel Hydrate Mono Hydrochloride Form 2 Crystalline Polymorph

Solid Compound 1, Form 1 (for instance prepared as described in Examples 1A-1C) ground with mortar and pestle (24.7 mg) was added to an HPLC vial with a stirbar. Methanol (0.156 mL) and water (0.243 mL) was added to the vial. The mixture was stirred at room temperature. After stirring for 17 days, the solid Form 2 was collected with centrifuge filtration.

### Example 2C: Preparation of Compound 1 Channel Hydrate Mono Hydrochloride Form 2 Crystalline Polymorph

Solid Compound 1, Form 1 (for instance prepared as described in Examples 1A- 1C) ground with mortar and pestle (22.0 mg) was added to an HPLC vial with a stirbar. Ethanol (0.252 mL) and water (0.154 mL) was added to the vial. The mixture was stirred at room temperature. After stirring for 17 days, the solid Form 2 was collected with centrifuge filtration.

### Example 3: PXRD Characterization of Polymorphic Form 1, Compound 1 Anhydrous Mono Hydrochloride

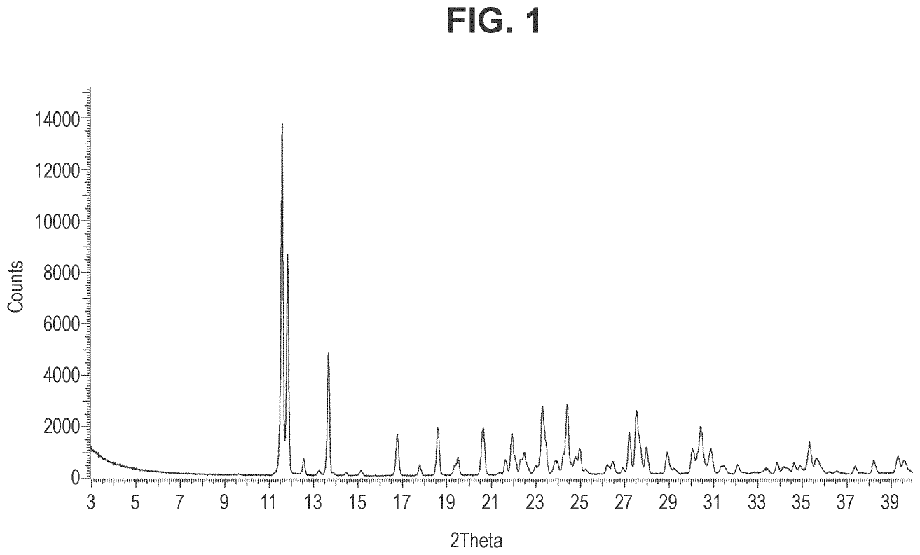

Powder X-ray diffraction analysis was conducted using a Bruker AXS D8 Advance diffractometer equipped with a Cu radiation source. Diffracted radiation was detected by a LYNXEYE_EX detector with motorized slits. Both primary and secondary equipped with 2.5 soller slits. The X-ray tube voltage and amperage were set at 40 kV and 40 mA respectively. Data was collected in the Theta-Theta goniometer in a locked couple scan at Cu K-alpha wavelength (CuKα̅=1.5418 λ) from 3.0 to 40.0 degrees 2-Theta with an increment of 0.01 degrees, using a scan speed of 1.0 seconds per step. Note that Cu K-beta wavelength was filtered. Samples were prepared by placement in a silicon low background sample holder and rotated during collection. Data were collected using Bruker DIFFRAC Plus software. Analysis performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked and peak positions were adjusted to the peak maximum. Peaks with relative intensity of ≥3% were generally chosen. The peaks which were not resolved or were consistent with noise were not selected. A typical error associated with the peak position from PXRD stated in USP up to +/−0.2° 2-Theta (USP-941). Crystalline Form 1 of Compound 1 was characterized by the PXRD pattern shown in FIG. 1. The PXRD pattern of Form 1, expressed in terms of the degree (2θ) and relative intensities with a relative intensity of ≥3.0%, measured on a Bruker AXS D8 Advance diffractometer with CuKα radiation, is also shown in Table 1:

TABLE 1A

| Angle | Relative |
|---|---|
| (2 theta) | Intensity (%) |
| 11.6 | 100 |
| 11.8 | 62.6 |
| 12.5 | 4.7 |
| 13.7 | 34.9 |
| 16.8 | 10.9 |
| 18.6 | 13.4 |
| 19.5 | 5.2 |
| 20.6 | 13.3 |
| 21.6 | 3.7 |
| 21.9 | 11.5 |
| 22.0 | 5.3 |
| 22.3 | 4.0 |
| 22.5 | 6.0 |
| 23.3 | 19.2 |
| 23.9 | 3.5 |
| 24.3 | 5.4 |
| 24.4 | 19.4 |
| 24.8 | 4.6 |
| 25.0 | 7.2 |
| 26.5 | 3.4 |
| 27.2 | 11.7 |
| 27.5 | 17.7 |
| 28.0 | 7.6 |
| 28.9 | 5.7 |
| 30.1 | 6.6 |
| 30.4 | 13.5 |
| 30.9 | 7.0 |
| 33.9 | 3.1 |
| 35.3 | 8.8 |
| 35.6 | 4.2 |
| 38.2 | 3.6 |
| 39.3 | 4.8 |
| 39.6 | 3.5 |

(Note that relative intensities may change depending on the crystal size and morphology.)

### Example 4: Single Crystal XRD Characterization of Polymorphic Form 1, Compound 1 Anhydrous Mono Hydrochloride

Good quality single crystals (0.2×0.04×0.02 mm3) were obtained from ethanol/water system utilizing slow solvent evaporation technique. Form 1 single crystal X-ray diffraction data collection was performed on a Bruker D8 Quest diffractometer at room temperature. Data collection consisted of omega and phi scans. The structure was solved by direct methods using SHELX software suite. The structures were subsequently refined by the full-matrix least squares method. All non-hydrogen atoms were found and refined using anisotropic displacement parameters. The hydrogen atoms located on nitrogen and oxygen were found from the Fourier difference map and refined with distances restrained. The remaining hydrogen atoms were placed in calculated positions and were allowed to ride on their carrier atoms. The final refinement included isotropic displacement parameters for all hydrogen atoms. A final difference Fourier revealed no missing or misplaced electron density. Pertinent crystal, data collection and refinement are summarized in Table 1B:

TABLE 1B

| | |
|---|---|
| Empirical formula | C₂₃ H₂₅ N₄ O₃ F Cl₂ |
| Formula weight | 495.37 |
| Temperature | 296(2) K |
| Wavelength | 1.54178 Å |
| Crystal system | Monoclinic |
| Space group | P2₁/c |
| Unit cell dimensions | α = 8.2310(2) Å, β = 36.6505(9) Å, χ = 8.1638(2) Å |
| | α = 90°, β = 111.973(2)°, γ = 90° |
| Volume | 2283.88(10) Å³ |
| Z | 4 |
| Theta range for data | 2.411 to 70.539° |
| collection | |
| Density (calculated) | 1.441 Mg/m³ |
| Completeness to theta = | 100.0% |
| 67.679° | |
| Absorption correction | Empirical |
| Goodness-of-fit on F² | 1.041 |
| Final R indices [I > 2sigma(I)] | R1 = 0.0553, wR2 = 0.1165 |
| R indices (all data) | R1 = 0.0840, wR2 = 0.1293 |

### Crystal Data and Structure Refinement for PF-06952229-01, Form 1 Anhydrous Mono Hydrochloride Salt

### Example 5: Solid State ¹³CNMR Characterization of Polymorphic Form 1, Compound 1 Anhydrous Mono Hydrochloride

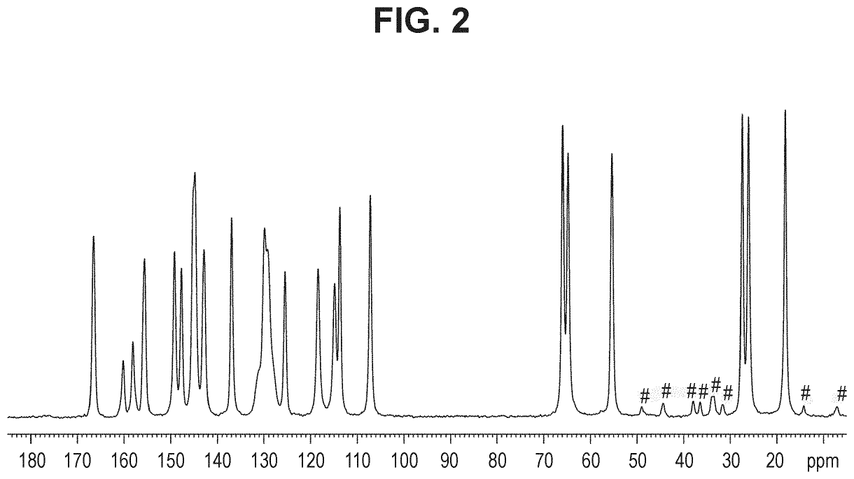

Solid state NMR (ssNMR) analysis was conducted on a CPMAS probe positioned into a Bruker-BioSpin Avance III 500 MHz (¹H frequency) NMR spectrometer. Material was packed into a 4 mm rotor sealed with an o-ring drive cap. The ¹³C ssNMR spectrum was collected using a proton decoupled cross-polarization magic angle spinning (CPMAS) experiment using a magic angle spinning rate of 14.0 kHz. The cross-polarization contact time was set to 2 ms and the recycle delay to 5 seconds. A phase modulated proton decoupling field of 80-90 kHz was applied during spectral acquisition. The number of scans was adjusted to obtain an adequate signal to noise ratio; 1024 scans were collected for API sample and 10240 scan collected for drug product samples. The ¹³C chemical shift scale was referenced using a ¹³C CPMAS experiment on an external standard of crystalline adamantane, setting its up-field resonance to 29.5 ppm (as determined from neat TMS). Automatic peak picking was performed using Bruker-BioSpin TopSpin version 3.5 software. Generally, a threshold value of 3% relative intensity was used for preliminary peak selection. The output of the automated peak picking was visually checked to ensure validity and adjustments were manually made if necessary. Although specific solid state NMR peak values are reported herein, there does exist a range for these peak values due to differences in instruments, samples, and sample preparation. This is common practice in the art of solid state NMR because of the variation inherent in peak positions. A typical variability for a ¹³C chemical shift x-axis value is on the order of plus or minus 0.2 ppm for a crystalline solid. The solid state NMR peak heights reported herein are relative intensities. Solid state NMR intensities can vary depending on the actual setup of the CPMAS experimental parameters and the thermal history of the sample. Crystalline Form 1 of Compound 1 was also characterized by the solid state ¹³C NMR spectral pattern shown in FIG. 2. The ¹³C chemical shifts of Form 1 of Compound 1 are shown in Table 2:

TABLE 2

| ¹³C Chemical Shifts | |
|---|---|
| [ppm] | Intensity |
| 18.2 | 100 |
| 26.1 | 98 |
| 27.5 | 99 |
| 55.4 | 86 |
| 64.8 | 86 |
| 66.0 | 95 |
| 107.2 | 72 |
| 113.8 | 68 |
| 114.9 | 43 |
| 118.4 | 48 |
| 125.5 | 47 |
| 129.2 | 54 |
| 129.8 | 62 |
| 136.9 | 65 |
| 142.8 | 54 |
| 144.8 | 80 |
| 145.1 | 75 |
| 147.7 | 48 |
| 149.2 | 54 |
| 155.6 | 51 |
| 158.1 | 24 |
| 160.2 | 18 |
| 166.6 | 59 |

### Example 6: Solid State ¹⁹FNMR Characterization of Polymorphic Form 1, Compound 1 Anhydrous Mono Hydrochloride

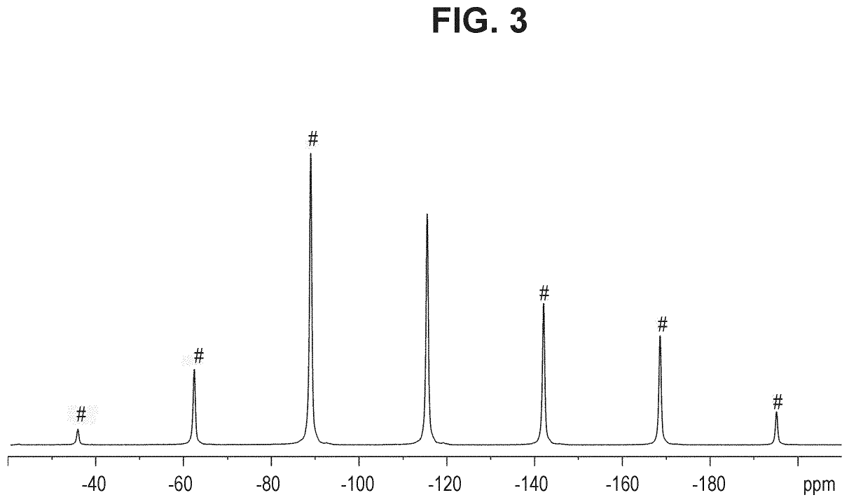

Solid state NMR (ssNMR) analysis was conducted on a CPMAS probe positioned into a Bruker-BioSpin Avance III 500 MHz (1H frequency) NMR spectrometer. Material was packed into a 4 mm rotor sealed with an o-ring drive cap. The 19F ssNMR spectrum was collected using a proton decoupled magic angle spinning (MAS) experiment using a magic angle spinning rate of 12.5 kHz. A phase modulated proton decoupling field of 80-90 kHz was applied during spectral acquisition. 256 scans were collected with a recycle delay of 25 s. The 19F chemical shift scale was referenced using a 19 F MAS experiment on an external standard of trifluoroacetic acid and water (50/50 volume/volume), setting its resonance to −76.5 ppm (as determined from neat TMS). Automatic peak picking was performed using Bruker-BioSpin TopSpin version 3.5 software. Generally, a threshold value of 3% relative intensity was used for preliminary peak selection. The output of the automated peak picking was visually checked to ensure validity and adjustments were manually made if necessary. Although specific solid state NMR peak values are reported herein, there does exist a range for these peak values due to differences in instruments, samples, and sample preparation. This is common practice in the art of solid state NMR because of the variation inherent in peak positions. A typical variability for a 19F chemical shift x-axis value is on the order of plus or minus 0.2 ppm for a crystalline solid. The solid state NMR peak heights reported herein are relative intensities. Solid state NMR intensities can vary depending on the actual setup of the CPMAS experimental parameters and the thermal history of the sample. Crystalline Form 1 of Compound 1 was characterized by the solid state ¹⁹F NMR spectral pattern shown in FIG. 3. The ¹⁹F chemical shifts of Form 1 of Compound 1 are shown in Table 3:

TABLE 3

| ¹⁹F Chemical Shifts | |
|---|---|
| [ppm] | Intensity |
| −115.6 | 100 |

### Example 7: Raman Characterization of Polymorphic Form 1, Compound 1 Anhydrous Mono Hydrochloride

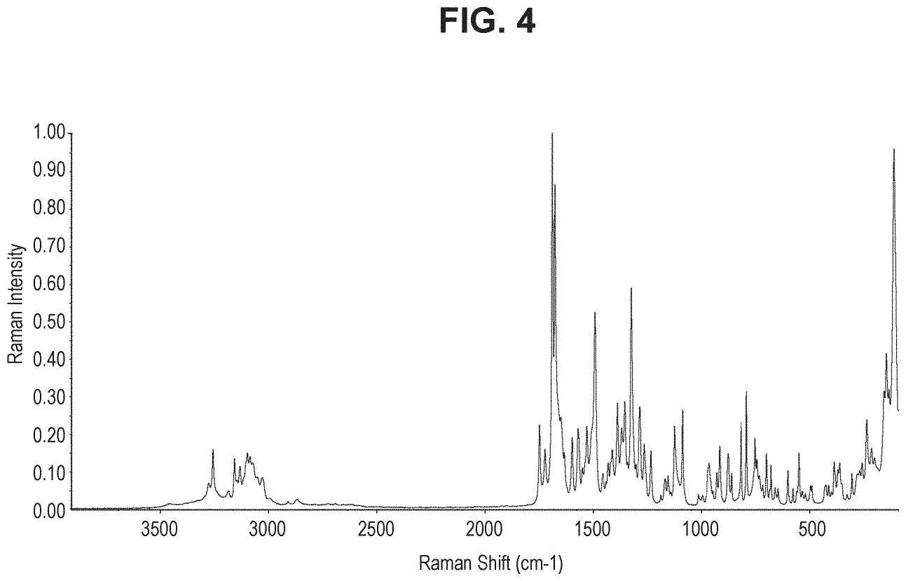

Raman spectra were collected using a Nicolet NXR FT-Raman accessory attached to the FT-IR bench. The spectrometer is equipped with a 1064 nm Nd:YVO4 laser and a liquid nitrogen cooled Germanium detector. Prior to data acquisition, instrument performance and calibration verifications were conducted using polystyrene. API samples were analyzed in glass NMR tubes that were static during spectral collection. The spectra were collected using 0.5 W of laser power and 512 co-added scans. The collection range was 3700-100 cm⁻¹. These spectra were recorded using 2 cm⁻¹ resolution and Happ-Genzel apodization. Utilizing the Raman method above, the possible variability associated with a spectral measurement is ±2 cm⁻¹. The intensity scale was normalized to 1 prior to peak picking. Peaks were manually identified using the Thermo Nicolet Omnic 9.7.46 software. Peak position was picked at the peak maximum, and peaks were only identified as such, if there was a slope on each side; shoulders on peaks were not included. For neat Form 1 API an absolute threshold of 0.016 with a sensitivity of 78 was utilized during peak picking. The peak position has been rounded to the nearest whole number using standard practice (0.5 rounds up, 0.4 rounds down). Peaks with normalized peak intensity between (1-0.75), (0.74-0.30), (0.29-0) were labeled as strong (S), medium (M) and weak (W), respectively. The relative peak intensity values are also illustrated in this report. Crystalline Form 1 of Compound 1 was also characterized by the following Raman spectral pattern, provided in FIG. 4. The Raman spectral peaks of Form 1 of Compound 1 are shown in Table 4:

TABLE 4

| Peak | | |
|---|---|---|
| position | Normalized | |
| (cm⁻¹) | intensity | Classification |
| 117 | 0.96 | S |
| 139 | 0.32 | M |
| 150 | 0.42 | M |
| 159 | 0.32 | M |
| 202 | 0.14 | W |
| 215 | 0.16 | W |
| 236 | 0.24 | W |
| 256 | 0.12 | W |
| 267 | 0.10 | W |
| 300 | 0.09 | W |
| 321 | 0.04 | W |
| 354 | 0.12 | W |
| 361 | 0.10 | W |
| 378 | 0.13 | W |
| 402 | 0.06 | W |
| 414 | 0.06 | W |
| 475 | 0.06 | W |
| 481 | 0.06 | W |
| 504 | 0.04 | W |
| 517 | 0.05 | W |
| 531 | 0.15 | W |
| 557 | 0.05 | W |
| 579 | 0.10 | W |
| 621 | 0.05 | W |
| 636 | 0.06 | W |
| 653 | 0.12 | W |
| 673 | 0.15 | W |
| 689 | 0.06 | W |
| 704 | 0.09 | W |
| 715 | 0.13 | W |
| 722 | 0.19 | W |
| 760 | 0.32 | M |
| 783 | 0.23 | W |
| 824 | 0.09 | W |
| 840 | 0.15 | W |
| 876 | 0.17 | W |
| 889 | 0.10 | W |
| 922 | 0.12 | W |
| 952 | 0.04 | W |
| 969 | 0.04 | W |
| 1038 | 0.27 | W |
| 1073 | 0.22 | W |
| 1102 | 0.09 | W |
| 1115 | 0.08 | W |
| 1133 | 0.04 | W |
| 1176 | 0.15 | W |
| 1205 | 0.17 | W |
| 1224 | 0.27 | W |
| 1242 | 0.12 | W |
| 1262 | 0.59 | M |
| 1279 | 0.13 | W |
| 1290 | 0.29 | W |
| 1304 | 0.22 | W |
| 1322 | 0.28 | W |
| 1344 | 0.16 | W |
| 1362 | 0.12 | W |
| 1385 | 0.09 | W |
| 1420 | 0.53 | W |
| 1455 | 0.22 | W |
| 1475 | 0.11 | W |
| 1493 | 0.21 | W |
| 1519 | 0.19 | W |
| 1554 | 0.15 | W |
| 1570 | 0.24 | W |
| 1594 | 0.88 | S |
| 1606 | 1.00 | S |
| 1637 | 0.16 | W |
| 1661 | 0.23 | W |
| 2715 | 0.02 | W |
| 2869 | 0.08 | W |
| 2923 | 0.14 | W |
| 2935 | 0.15 | W |
| 2967 | 0.11 | W |
| 2991 | 0.14 | W |
| 3017 | 0.05 | W |
| 3085 | 0.16 | W |
| 3104 | 0.07 | W |

### Example 8: PXRD Characterization of Polymorphic Form 2, Compound 1 Channel Hydrate Mono Hydrochloride

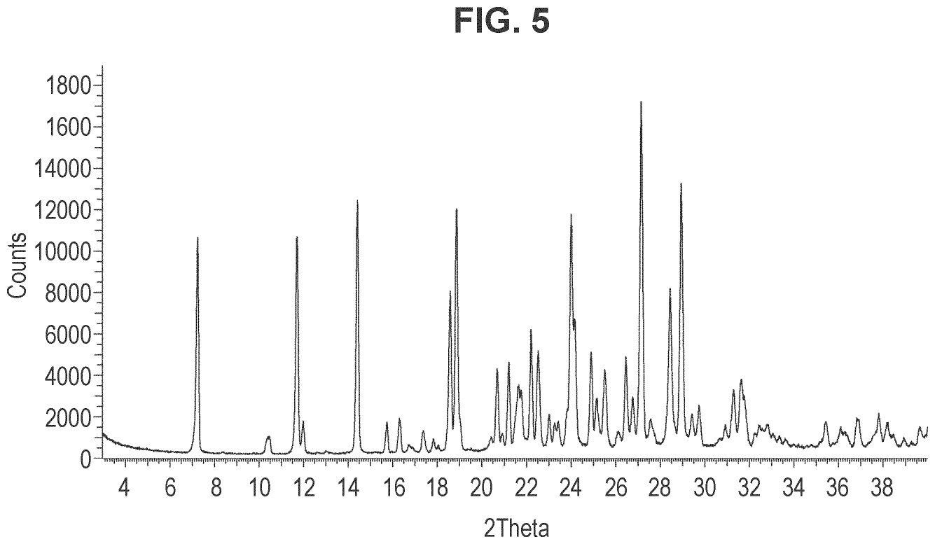

Powder X-ray diffraction analysis was conducted using a Bruker AXS D8 Advance diffractometer equipped with a Cu radiation source. Diffracted radiation was detected by a LYNXEYE_EX detector with motorized slits. Both primary and secondary equipped with 2.5 soller slits. The X-ray tube voltage and amperage were set at 40 kV and 40 mA respectively. Data was collected in the Theta-Theta goniometer in a locked couple scan at Cu K-alpha wavelength (CuKα̅=1.5418 λ) from 3.0 to 40.0 degrees 2-Theta with an increment of 0.01 degrees, using a scan speed of 1.0 seconds per step. Note that Cu K-beta wavelength was filtered. Samples were prepared by placement in a silicon low background sample holder and rotated during collection. Data were collected using Bruker DIFFRAC Plus software. Analysis performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked and peak positions were adjusted to the peak maximum. Peaks with relative intensity of 3% were generally chosen. The peaks which were not resolved or were consistent with noise were not selected. A typical error associated with the peak position from PXRD stated in USP up to +/−0.2° 2-Theta (USP-941). Crystalline Form 2 of Compound 1 was characterized by the PXRD pattern shown in FIG. 5. The PXRD pattern of Form 2, expressed in terms of the degree (2θ) and relative intensities with a relative intensity of ≤3.0%, is also shown in Table 5:

TABLE 5A

| Angle | Relative |
|---|---|
| (2 theta) | Intensity (%) |
| 7.2 | 62.9 |
| 10.4 | 4.6 |
| 11.7 | 64.4 |
| 12.0 | 8.6 |
| 14.4 | 74.5 |
| 15.7 | 8.7 |
| 16.3 | 9.8 |
| 17.4 | 6.4 |
| 17.8 | 3.9 |
| 18.6 | 47.2 |
| 18.9 | 71.8 |
| 20.4 | 3.6 |
| 20.7 | 23.3 |
| 20.9 | 4.1 |
| 21.2 | 23.6 |
| 21.7 | 17.7 |
| 22.2 | 33.5 |
| 22.5 | 27.9 |
| 23.0 | 9.2 |
| 23.3 | 6.3 |
| 23.4 | 6.7 |
| 23.8 | 10.6 |
| 24.0 | 67.3 |
| 24.1 | 36.7 |
| 24.9 | 27.5 |
| 25.2 | 14.1 |
| 25.5 | 22.0 |
| 26.1 | 4.4 |
| 26.5 | 25.8 |
| 26.8 | 14.5 |
| 27.2 | 100 |
| 27.6 | 7.6 |
| 28.4 | 46.0 |
| 29.0 | 76.3 |
| 29.4 | 9.2 |
| 29.7 | 11.9 |
| 30.9 | 6.1 |
| 31.3 | 16.4 |
| 31.6 | 19.5 |
| 31.8 | 14.9 |
| 32.3 | 3.5 |
| 32.5 | 5.8 |
| 32.8 | 6.2 |
| 33.1 | 3.2 |
| 35.4 | 7.4 |
| 36.1 | 5.9 |
| 36.3 | 4.2 |
| 36.9 | 7.5 |
| 37.8 | 9.2 |
| 38.2 | 6.6 |
| 38.9 | 3.0 |
| 39.7 | 4.2 |

(Note that relative intensities may change depending on the crystal size and morphology.)

### Example 9: Solid State ¹³CNMR Characterization of Polymorphic Form 2, Compound 1 Channel Hydrate Mono Hydrochloride

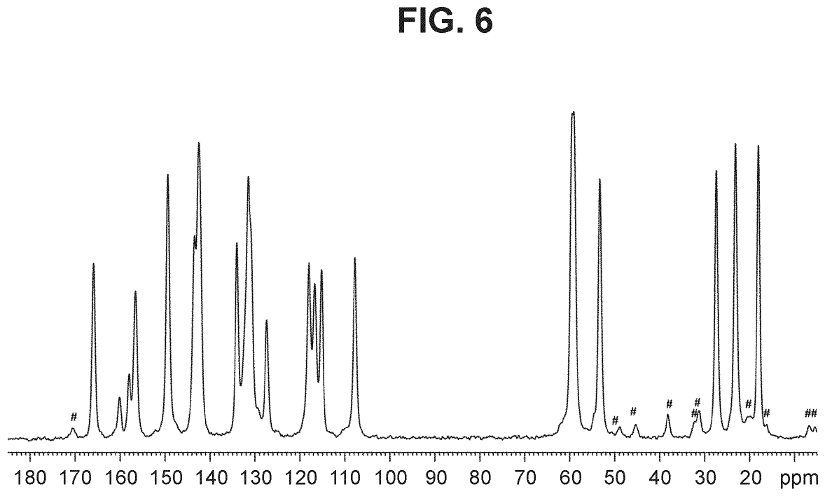

Solid state NMR (ssNMR) analysis was conducted on a CPMAS probe positioned into a Bruker-BioSpin Avance III 500 MHz (¹H frequency) NMR spectrometer. Material was packed into a 4 mm rotor sealed with an o-ring drive cap. The ¹³C ssNMR spectrum was collected using a proton decoupled cross-polarization magic angle spinning (CPMAS) experiment using a magic angle spinning rate of 14.0 kHz. The cross-polarization contact time was set to 2 ms and the recycle delay to 5 seconds. A phase modulated proton decoupling field of 80-90 kHz was applied during spectral acquisition. The number of scans was adjusted to obtain an adequate signal to noise ratio; 1024 scans were collected for API sample and 10240 scan collected for drug product samples. The ¹³C chemical shift scale was referenced using a ¹³C CPMAS experiment on an external standard of crystalline adamantane, setting its up-field resonance to 29.5 ppm (as determined from neat TMS). Automatic peak picking was performed using Bruker-BioSpin TopSpin version 3.5 software. Generally, a threshold value of 3% relative intensity was used for preliminary peak selection. The output of the automated peak picking was visually checked to ensure validity and adjustments were manually made if necessary. Although specific solid state NMR peak values are reported herein, there does exist a range for these peak values due to differences in instruments, samples, and sample preparation. This is common practice in the art of solid state NMR because of the variation inherent in peak positions. A typical variability for a ¹³C chemical shift x-axis value is on the order of plus or minus 0.2 ppm for a crystalline solid. The solid state NMR peak heights reported herein are relative intensities. Solid state NMR intensities can vary depending on the actual setup of the CPMAS experimental parameters and the thermal history of the sample. Crystalline Form 2 of Compound 1 was characterized by the solid state ¹³C NMR spectral pattern shown in FIG. 6, carried out on a CPMAS probe positioned into a Bruker-Biospin Avance III 500 MHz NMR spectrometer. The ¹³C chemical shifts of Form 2 of Compound 1 are shown in Table 6:

TABLE 6

| ¹³C Chemical Shifts | |
|---|---|
| [ppm] | Intensity |
| 18.0 | 89 |
| 23.2 | 90 |
| 27.4 | 82 |
| 53.3 | 79 |
| 59.2 | 100 |
| 59.5 | 100 |
| 107.8 | 55 |
| 115.2 | 51 |
| 116.7 | 47 |
| 118.0 | 54 |
| 127.4 | 36 |
| 131.0 | 66 |
| 131.5 | 80 |
| 134.0 | 60 |
| 142.5 | 91 |
| 143.4 | 62 |
| 149.4 | 81 |
| 156.6 | 45 |
| 158.0 | 20 |
| 160.1 | 12 |
| 165.9 | 54 |

### Example 10: Solid State ¹⁹FNMR Characterization of Polymorphic Form 2, Compound 1 Channel Hydrate Mono Hydrochloride

Solid state NMR (ssNMR) analysis was conducted on a CPMAS probe positioned into a

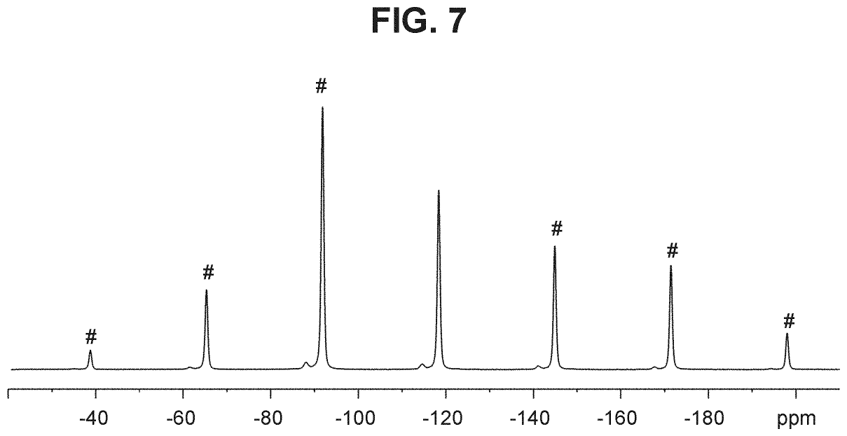

Bruker-BioSpin Avance III 500 MHz (1 H frequency) NMR spectrometer. Material was packed into a 4 mm rotor sealed with an o-ring drive cap. The 19 F ssNMR spectrum was collected using a proton decoupled magic angle spinning (MAS) experiment using a magic angle spinning rate of 12.5 kHz. A phase modulated proton decoupling field of 80-90 kHz was applied during spectral acquisition. 256 scans were collected with a recycle delay of 25 s. The 19 F chemical shift scale was referenced using a 19F MAS experiment on an external standard of trifluoroacetic acid and water (50/50 volume/volume), setting its resonance to −76.5 ppm (as determined from neat TMS). Automatic peak picking was performed using Bruker-BioSpin TopSpin version 3.5 software. Generally, a threshold value of 3% relative intensity was used for preliminary peak selection. The output of the automated peak picking was visually checked to ensure validity and adjustments were manually made if necessary. Although specific solid state NMR peak values are reported herein, there does exist a range for these peak values due to differences in instruments, samples, and sample preparation. This is common practice in the art of solid state NMR because of the variation inherent in peak positions. A typical variability for a 19 F chemical shift x-axis value is on the order of plus or minus 0.2 ppm for a crystalline solid. The solid state NMR peak heights reported herein are relative intensities. Solid state NMR intensities can vary depending on the actual setup of the CPMAS experimental parameters and the thermal history of the sample. Crystalline Form 2 of Compound 1 was also characterized by the solid state ¹⁹F NMR spectral pattern shown in FIG. 7, carried out on a CPMAS probe positioned into a Bruker-Biospin Avance III 500 MHz NMR spectrometer. The ¹⁹F chemical shifts of Form 2 of Compound 1 are shown in Table 7:

TABLE 7

| ¹⁹F Chemical Shifts | |
|---|---|
| [ppm] | Intensity |
| −118.5 | 100 |
| −114.7 | 3 |

### Example 11: Raman Characterization of Polymorphic Form 2, Compound 1 Channel Hydrate Mono Hydrochloride

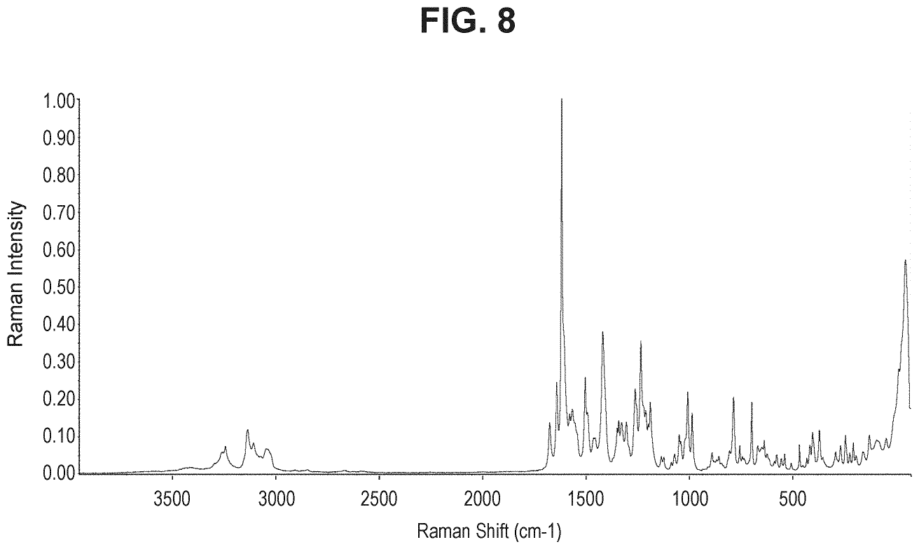

Raman spectra were collected using a Nicolet NXR FT-Raman accessory attached to the FT-IR bench. The spectrometer is equipped with a 1064 nm Nd:YVO4 laser and a liquid nitrogen cooled Germanium detector. Prior to data acquisition, instrument performance and calibration verifications were conducted using polystyrene. API samples were analyzed in glass NMR tubes that were static during spectral collection. The spectra were collected using 0.5 W of laser power and 512 co-added scans. The collection range was 3700-100 cm⁻¹. These spectra were recorded using 2 cm⁻¹ resolution and Happ-Genzel apodization. Utilizing the Raman method above, the possible variability associated with a spectral measurement is ±2 cm⁻¹. The intensity scale was normalized to 1 prior to peak picking. Peaks were manually identified using the Thermo Nicolet Omnic 9.7.46 software. Peak position was picked at the peak maximum, and peaks were only identified as such, if there was a slope on each side; shoulders on peaks were not included. For neat Form 1 API an absolute threshold of 0.016 with a sensitivity of 78 was utilized during peak picking. The peak position has been rounded to the nearest whole number using standard practice (0.5 rounds up, 0.4 rounds down). Peaks with normalized peak intensity between (1-0.75), (0.74-0.30), (0.29-0) were labeled as strong (S), medium (M) and weak (W), respectively. The relative peak intensity values are also illustrated in this report. Crystalline Form 2 of Compound 1 was also characterized by the following Raman spectral pattern, provided in FIG. 8, carried out on a Nicolet NXR FT-Raman accessory attached to the FT-IR bench. The spectrometer is equipped with a 1064 nm Nd:YVO4 laser and a liquid nitrogen cooled Germanium detector. The Raman spectral peaks of Form 2 of Compound 1 are shown in Table 8:

TABLE 8

| Peak | | |
|---|---|---|
| position | Normalized | |
| (cm⁻¹) | intensity | Classification |
| 119 | 0.57 | M |
| 202 | 0.09 | W |
| 243 | 0.09 | W |
| 276 | 0.10 | W |
| 304 | 0.06 | W |
| 333 | 0.05 | W |
| 345 | 0.08 | W |
| 360 | 0.05 | W |
| 379 | 0.10 | W |
| 400 | 0.07 | W |
| 421 | 0.06 | W |
| 493 | 0.12 | W |
| 522 | 0.11 | W |
| 533 | 0.08 | W |
| 547 | 0.04 | W |
| 578 | 0.08 | W |
| 615 | 0.03 | W |
| 642 | 0.05 | W |
| 657 | 0.04 | W |
| 676 | 0.05 | W |
| 719 | 0.05 | W |
| 732 | 0.09 | W |
| 760 | 0.07 | W |
| 786 | 0.19 | W |
| 826 | 0.05 | W |
| 837 | 0.07 | W |
| 864 | 0.20 | W |
| 928 | 0.05 | W |
| 958 | 0.06 | W |
| 1044 | 0.16 | W |
| 1063 | 0.22 | W |
| 1100 | 0.10 | W |
| 1119 | 0.05 | W |
| 1134 | 0.03 | W |
| 1166 | 0.04 | W |
| 1178 | 0.05 | W |
| 1225 | 0.19 | W |
| 1234 | 0.13 | W |
| 1246 | 0.17 | W |
| 1266 | 0.36 | M |
| 1290 | 0.23 | W |
| 1328 | 0.14 | W |
| 1349 | 0.14 | W |
| 1362 | 0.14 | W |
| 1369 | 0.12 | W |
| 1431 | 0.38 | M |
| 1465 | 0.10 | W |
| 1508 | 0.26 | W |
| 1563 | 0.17 | W |
| 1575 | 0.16 | W |
| 1609 | 1.00 | S |
| 1631 | 0.24 | W |
| 1662 | 0.14 | W |
| 2890 | 0.07 | W |
| 2946 | 0.08 | W |
| 2970 | 0.12 | W |
| 3068 | 0.07 | W |

### Example 12: Comparison of Critical Water Activity of Free Base Forms of Compound 1 and Mono-Hydrochloride Forms (Forms 1 and 2) of Compound 1

Critical water activity is a phase boundary above which a hydrate is a stable form. On the other hand, below the critical water activity, the anhydrous form is more stable as compared to its hydrated form (a_{w}). Values of a_{w}. correspond to percent humidity, where the percent humidity is described as 100 x a_{w}. Thus 0.1 a_{w} corresponds to 10% humidity, 0.2 a_{w} corresponds to 20% humidity, and so on.

The claimed polymorphic forms of Compound 1 mono-hydrochloride interconvert between Form 1 and Form 2 at a critical water activity of between 0.60 and 0.65 (corresponding to a relative humidity of between 60% and 65%). The critical water activity of the free base form of Compound 1 is between 0.2 and 0.3 (corresponding to a relative humidity between 20% and 30%). See Table 9. It is noted that manufacturing processes are routinely run at relative humidities that exceed 30% relative humidity, but manufacturing processes run at relative humidities of >60% are unusual.

TABLE 9

| | Water | | | |
|---|---|---|---|---|
| | Activity at | | | |
| | Room | | | |
| Solid Form | Temperature | | PXRD Result | Conclusion |
| Compound 1, | 0.1 | a_{w} | free base anhydrous | Critical water activity |
| Free Base | 0.2 | a_{w} | free base anhydrous | between 0.2 and 0.3 Aw |
| | 0.3 | a_{w} | free base monohydrate | |
| | 0.5 | a_{w} | free base monohydrate | |
| | 1.0 | Aw | free base monohydrate | |
| Compound 1, | 0.60 | a_{w} | Form 1 | Critical water activity |
| mono-HCl salt | 0.65 | a_{w} | Form 2 | between 0.60 and 0.65 |
| | 0.70 | a_{w} | Form 2 | Aw |
| | 0.75 | a_{w} | Form 2 | |
| | 0.80 | a_{w} | Form 2 | |

Samples were tested using powder X-ray diffraction. Analysis was conducted using a Bruker AXS D8 Endeavor diffractometer equipped with a Cu radiation source. The divergence slit was set at 6 mm continuous illumination. Diffracted radiation was detected by a PSD-Lynx Eye detector, with the detector PSD opening set at 4.104 degrees. The X-ray tube voltage and amperage were set to 40 kV and 40 mA respectively. Data was collected in the Theta-Theta goniometer at the Cu wavelength from 3.0 to 40.0 degrees 2-Theta using a step size of 0.020 degrees and a step time of 0.3 second. Samples were prepared by placing them in a silicon low background sample holder and rotated during collection. Data were collected using Bruker DIFFRAC Plus software and analysis was performed by EVA diffract plus software.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

We claim:

1. A crystalline form of 4-(2-(5-chloro-2-fluorophenyl)-5-isopropylpyridin-4-ylamino)-N-(1,3-dihydroxypropan-2-yl)nicotinamide mono hydrochloride.

2. A compound of claim 1 which is anhydrous.

3. A compound of claim 1 which is a hydrate.

4. An anhydrous crystalline form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a powder X-ray diffraction pattern comprising peaks at diffraction angle (2 degrees θ) of 13.7±0.2 and 24.4±0.2.

5. An anhydrous crystalline form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a Raman spectrum comprising Raman shift peaks (cm−1) at 1594±2 cm⁻¹, 1606±2 cm⁻¹ and 1637±2 cm⁻¹.

6. The crystalline form of claim 5, wherein said crystalline form has a Raman spectrum comprising Raman shift peaks (cm−1) at 876±2 cm⁻¹, 1519±2 cm⁻¹, 1594±2 cm⁻¹, 1606±2 cm⁻¹ and 1637±2 cm⁻¹.

7. An anhydrous crystalline form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a solid state NMR spectrum comprising ¹³C chemical shifts at 136.9±0.2, 26.1±0.2 and 147.7±0.2 ppm.

8. The crystalline form of claim 7, wherein said crystalline form has a solid state NMR spectrum comprising ¹³C chemical shifts at 136.9±0.2, 26.1±0.2, 147.7±0.2, 125.5±0.2 and 55.4±0.2 ppm.

9. An anhydrous crystalline form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a solid state NMR spectrum comprising an ¹⁹F chemical shift at −115.6±0.2 ppm.

10. An anhydrous crystalline form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a powder X-ray diffraction pattern comprising peaks at positions essentially the same as shown in FIG. 1.

11. An anhydrous crystalline form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a Raman spectrum comprising Raman shift peaks (cm−1) at positions essentially the same as shown in FIG. 4.

12. An anhydrous crystalline form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a solid state NMR spectrum comprising ¹³C chemical shifts at positions essentially the same as shown in FIG. 2.

13. An anhydrous crystalline form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a solid state NMR spectrum comprising an ¹⁹F chemical shift at position(s) essentially the same as shown in FIG. 3.

14. The crystalline form of claim 4, wherein said crystalline form additionally has a Raman spectrum comprising Raman shift peaks (cm−1) at at least one of 1594±2 cm⁻¹, 1606±2 cm⁻¹ and 1637±2 cm⁻¹.

15. The crystalline form of claim 4, wherein said crystalline form additionally has a Raman spectrum comprising Raman shift peaks (cm−1) at positions essentially the same as shown in FIG. 4.

16. The crystalline form of claim 4, wherein said crystalline form additionally has a solid state NMR spectrum comprising ¹³C chemical shifts at at least one of 136.9±0.2, 26.1±0.2 and 147.7±0.2 ppm.

17. The crystalline form of claim 4, wherein said crystalline form additionally has a solid state NMR spectrum comprising ¹³C chemical shifts at positions essentially the same as shown in FIG. 2.

18. The crystalline form of claim 4, wherein said crystalline form additionally has a solid state NMR spectrum comprising an ¹⁹F chemical shift at −115.6±0.2 ppm.

19. The crystalline form of claim 4, wherein said crystalline form additionally has a solid state NMR spectrum comprising ¹⁹F chemical shifts at position(s) essentially the same as shown in FIG. 3.

20. An anhydrous crystalline form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a powder X-ray diffraction pattern comprising peaks at diffraction angle (2 degrees θ) of at least one of 13.7±0.2 and 24.4±0.2, and a solid state NMR spectrum comprising an ¹⁹F chemical shift at −115.6±0.2 ppm.

21. The crystalline form of claim 4 which is substantially pure 4-(2-(5-chloro-2-fluorophenyl) -5-isopropylpyridin-4-ylamino)-N-(1,3-dihydroxypropan-2-yl)nicotinamide mono hydrochloride.

22. The substantially pure crystalline form of claim 21, wherein said 4-(2-(5-chloro-2-fluorophenyl) -5-isopropylpyridin-4-ylamino)-N-(1,3-dihydroxypropan-2-yl)nicotinamide mono hydrochloride is at least 90% pure.

23. The substantially pure crystalline form of claim 21, wherein said 4-(2-(5-chloro-2-fluorophenyl) -5-isopropylpyridin-4-ylamino)-N-(1,3-dihydroxypropan-2-yl)nicotinamide mono hydrochloride is at least 95% pure.

24. The substantially pure crystalline form of claim 21, wherein said 4-(2-(5-chloro-2-fluorophenyl) -5-isopropylpyridin-4-ylamino)-N-(1,3-dihydroxypropan-2-yl)nicotinamide mono hydrochloride is at least 99% pure.

25. A method of treating cancer in a mammal, said method comprising administering to the mammal a therapeutically effective amount of the crystalline form of claim 4.

26. The method of claim 25, wherein said cancer is selected from the group consisting of mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, and a combination of one or more of the foregoing cancers.

27. A crystalline hydrate form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a powder X-ray diffraction pattern comprising peaks at diffraction angle (2 degrees θ) of 7.2±0.2, 15.7±0.2 and 18.9±0.2.

28. The crystalline form of claim 27, wherein said crystalline form has a powder X-ray diffraction pattern comprising peaks at diffraction angle (2 degrees θ) of 7.2±0.2, 15.7±0.2, 17.4±0.2, 18.9±0.2 and 28.4±0.2.

29. A crystalline hydrate form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a Raman spectrum comprising Raman shift peaks (cm−1) at 1508±2 cm⁻¹, 1609±2 cm^{-l}and 1631±2 cm⁻¹.

30. The crystalline form of claim 29, wherein said crystalline form has a Raman spectrum comprising Raman shift peaks (cm−1) at 1508±2 cm⁻¹, 1609±2 cm^{-l}and 1631±2 cm⁻¹, 864 and 786.

31. A crystalline hydrate form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a solid state NMR spectrum comprising ¹³C chemical shifts at 165.9±0.2, 53.3±0.2 and 23.2±0.2 ppm.

32. The crystalline form of claim 31, wherein said crystalline form has a solid state NMR spectrum comprising ¹³C chemical shifts at 165.9±0.2, 53.3±0.2 and 23.2±0.2, 115.2±0.2 and 156.6±0.2 ppm.

33. A crystalline hydrate form of 4-(2-(5-chloro-2-fluorophenyl)-5-isopropylpyridin-4-ylamino) -N-(1,3-dihydroxypropan-2-yl)nicotinamide mono hydrochloride, wherein said crystalline form has a solid state NMR spectrum comprising an ¹⁹F chemical shift at −118.5±0.2 ppm.

34. A pharmaceutical composition comprising the crystalline form of any of claims 1, 4 and 27.

35. The crystalline form of claim 27 which is substantially pure 4-(2-(5-chloro-2-fluorophenyl) -5-isopropylpyridin-4-ylamino)-N-(1,3-dihydroxypropan-2-yl)nicotinamide mono hydrochloride.

36. A method of treating cancer in a mammal, said method comprising administering to the mammal a therapeutically effective amount of the crystalline form of claim 27.

37. The method of claim 36, wherein said cancer is selected from the group consisting of mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, and a combination of one or more of the foregoing cancers.
